(19) United States
(12) Patent Application Publication
(10) Pub. No.: US 2025/0057864 A1
(43) Pub. Date: Feb. 20, 2025

(54) COMPOSITION FOR IMPROVING EXERCISE PERFORMANCE COMPRISING GYPENOSIDE COMPOUND AS ACTIVE INGREDIENT

(71) Applicant: BTC CORPORATION, Gyeonggi-do (KR)

(72) Inventors: Tae Young KIM, Gyeonggi-do (KR); Joo Myung MOON, Gyeonggi-do (KR); Yoon Hee KIM, Gyeonggi-do (KR)

(73) Assignee: BTC CORPORATION, Gyeonggi-do (JP)

(21) Appl. No.: 18/724,442

(22) PCT Filed: Jan. 13, 2023
(86) PCT No.: PCT/KR2023/000684

§ 371 Date: Jun. 26, 2024

(30) Foreign Application Priority Data
Jan. 14, 2022 (KR) 10-2022-0005747

(51) Int. Cl. A61K 31/704; A61P 21/00

(52) U.S. Cl. CPC A61K 31/704; A61P 21/00

(57) ABSTRACT

The present disclosure relates to a composition for improving exercise performance, or preventing, ameliorating or treating muscle diseases, the composition containing a gypenoside compounds as an active ingredient. A composition of the present disclosure, which contains a gypenoside compound as an active ingredient, has an excellent effect in improving exercise performance and enhancing physical strength and, thus, is expected to be very useful in the field of pharmaceuticals or functional foods.

### CROSS-REFERENCE TO RELATED APPLICATIONS

This application is a § 371 national stage entry of International Application No. PCT/KR2023/000684 filed on Jan. 13, 2023, which claims priority to Korean Patent Application No. 10-2022-0005747 filed on Jan. 14, 2022, the entire contents of which are incorporated herein by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Jun. 26, 2024, is named G1035-28301_SequenceListing and is 44,870 bytes in size.

### TECHNICAL FIELD

The present disclosure relates to a composition for improving exercise performance, which contains a gypenoside compound as an active ingredient.

### BACKGROUND ART

In the complex modern society, people who are exposed to worsening living conditions due to environmental pollution, mental stress, lack of activity, etc. are increasingly concerned about improving health. While exercise is the most effective and economical preventive measure against adult diseases or aging, modern people who are unable to take care of their health due to busy daily lives, fatigue, etc. are interested in taking a variety of functional foods as an alternative to exercise. Furthermore, in addition to scientific training and diets, athletes use ergogenic aids to increase exercise performance. The ergogenic aids are commonly used not only by athletes but also by the general public, because they not only improve exercise performance but also remove fatigue factors that are accumulated in the body during physical activities and cause fatigue.

Researches on functional adjuvants for improving exercise performance are being carried out around the world. Although the ergogenic aids that contain compounds such as steroids, caffeine, sodium carbonate, sodium citrate, etc. increase exercise performance and provide vitality in everyday lives, the effect is only temporary and there is a risk of causing deadly side effects on health.

Accordingly, there has recently been a great need for the development of functional supplements using safe natural substances or naturally derived compounds such as plant extracts.

The inventors of the present disclosure have studied on the effect of naturally derived compounds on improvement of exercise performance and amelioration or treatment of muscle diseases. As a result, they have found out that gypenoside compounds have excellent effect of improving exercise performance and completed the present disclosure.

### DISCLOSURE

### Technical Problem

The present disclosure is directed to providing a food composition for improving exercise performance, which contains a gypenoside compound as an active ingredient.

The present disclosure is also directed to providing a pharmaceutical composition for improving exercise performance, which contains a gypenoside compound as an active ingredient.

### Technical Solution

The present disclosure provides a health functional food composition for improving exercise performance, which contains a gypenoside compound represented by Chemical Formula 1, a stereoisomer thereof or a sitologically acceptable salt thereof as an active ingredient.

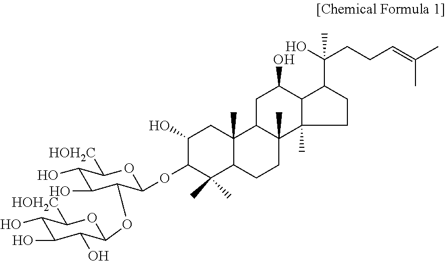

According to an exemplary embodiment of the present disclosure, the active ingredient may be gypenoside L or gypenoside LI.

According to an exemplary embodiment of the present disclosure, the active ingredient may be gypenoside L and gypenoside LI.

According to an exemplary embodiment of the present disclosure, the weight ratio of gypenoside L and gypenoside LI as the active component may be 100:20 to 80.

According to an exemplary embodiment of the present disclosure, the weight ratio of gypenoside L and gypenoside LI as the active component may be 100:30 to 70.

According to an exemplary embodiment of the present disclosure, the administration dosage of the active ingredient may be 0.01 to 200 mg/kg/day.

Furthermore, the present disclosure provides a pharmaceutical composition for improving exercise performance, which contains the gypenoside compound represented by Chemical Formula 1, a stereoisomer thereof or a pharmaceutically acceptable salt thereof as an active ingredient.

Furthermore, the present disclosure provides a health functional food composition for preventing or improving muscle diseases, which contains the gypenoside compound represented by Chemical Formula 1, a stereoisomer thereof or a sitologically acceptable salt thereof as an active ingredient.

### Advantageous Effects

A composition of the present disclosure, which contains a gypenoside compound as an active ingredient, has an excellent effect in improving exercise performance and enhancing physical strength and, thus, is expected to be very useful in the field of pharmaceuticals or functional foods.

In addition, the composition of the present disclosure, which contains a gypenoside compound as an active ingredient, has excellent effect in reducing ROS production and activating PGC-1α and AMPK involved in mitochondrial function within muscle. In addition, it activates Nrf2, which regulates the expression of antioxidant genes that can protect mitochondria from oxidative stress and inhibit muscle damage. In addition, it can be useful for improving exercise performance since it increases the expression of TFAM, CPT-1β and mtDNA involved in the replication of mitochondria within muscle and can enhance the expression of GSY, SIRT and PPARγ involved in change of muscle type and energy generation. Furthermore, it can significantly improve exercise performance by improving muscle fatigue, increasing exercise duration and exercise capacity and increasing glycogen content in muscle.

Since a naturally derived compound is used in the composition of the present disclosure as an active ingredient, it can be used safely without side effects, and can be usefully used as a pharmaceutical, a food, etc.

### BEST MODE

Hereinafter, the present disclosure is described in detail.

The term “exercise performance” or “exercise ability” used in this specification refers to the ability of performing physical activities in daily lives or sports, such as running, jumping, throwing, swimming, etc. quickly, strongly, accurately and skillfully for a long time. The exercise performance is defined by such factors as muscular strength, balance, motor coordination, agility, endurance, etc. The term “improvement of exercise performance” refers to the improvement or enhancement of exercise performance, and specifically, to the improvement or enhancement of endurance, balance or muscular strength.

The term “pharmaceutically acceptable salt” used in this specification refers to the form of a compound which does not cause serious irritation to an organism to which the compound is administered and does not damage the biological activity and physical properties of the compound.

The “pharmaceutically acceptable salt” includes, for example, an acid addition salt formed from the addition of an inorganic acid such as chloric acid, sulfuric acid, nitric acid, phosphoric acid, hydrobromic acid, hydroiodic acid, etc. or an organic acid such as tartaric acid, formic acid, citric acid, acetic acid, trichloroacetic acid, fluoroacetic acid, gluconic acid, benzoic acid, lactic acid, fumaric acid, maleic acid, salicylic acid, methanesulfonic acid, ethanesulfonic acid, benzenesulfonic acid, p-toluenesulfonic acid, etc. When a carboxylic acid group is present in the compound of Chemical Formula 1, examples of a pharmaceutically acceptable salt of the carboxylic acid include a metal salt or an alkaline earth metal salt formed by lithium, sodium, potassium, calcium, magnesium, etc., an amino acid salt of lysine, arginine, guanidine, etc., and an organic salt of dicyclohexylamine, N-methyl-D-glucamine, tris(hydroxymethyl)methylamine, diethanolamine, choline, triethylamine, etc. The compound of Chemical Formula 1 according to the present disclosure can be converted to its salt by conventional methods.

The term “stereoisomer” used in this specification refers to an isomer that has the same chemical or molecular formula but is formed through the spatial arrangement of atoms in the molecule. It is classified into “enantiomers” or “diastereoisomers”. The “enantiomer” refers to an isomer that does not overlap with its mirror image like the relationship between the right hand and the left hand, and the “diastereoisomer” refers to an isomer in which there is no optical inversion. The diastereoisomer includes geometric isomers with a non-rotatable bond such as a double bond, a conformational isomer with a temporarily different arrangement due to the rotation of a single bond, a common diastereoisomer with multiple stereocenters but does not form a mirror image, etc. All isomers and mixtures thereof are also included within the scope of the present disclosure.

The term “active ingredient” used in this specification refers to an ingredient which represents the intended activity on its own or can exhibit the activity together with a carrier that cannot exhibit the activity on its own.

The present disclosure provides a food composition for improving exercise performance, which contains a gypenoside compound, a stereoisomer thereof or a sitologically acceptable salt thereof as an active ingredient.

The gypenoside compounds may be a gypenoside L compound represented by Chemical Formula 1 (gypenoside 50).

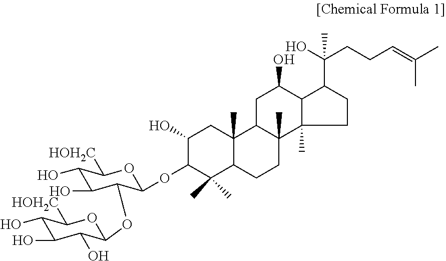

In the present disclosure, the “gypenoside L” is used interchangeably with “Gyp L”, “Gyp 50”, “G50” or “gypenoside 50”, which is a type of gypenoside (Gyp).

The gypenoside L can be synthesized chemically or may be isolated from a natural substance. When the gypenoside L of the present disclosure is isolated from a natural substance, it may include an extract of the natural substance or a fraction thereof, as long as it contains the gypenoside L.

Furthermore, since the compound of the present disclosure may have an asymmetric carbon center, it may exist as an R or S isomer, a racemate, a diastereoisomeric mixture or individual diastereoisomers, all of which are included within the scope of the present disclosure.

The term “gypenoside (Gyp)” used in this specification refers to a triterpenoid saponin. As the types of gypenoside, Gyp L, Gyp LI, Gyp LXXV, Gyp XVII, Gyp XLIX, Gyp XXIV, Gyp XLV, etc. are known.

The gypenoside L of the present disclosure may be obtained by hydroxylation from gypenoside Rg3 in accordance with known methods or may be isolated from a plant extract. Alternatively, it may be purchased from the market.

The term “hydroxylation” used in this specification refers to a reaction by which a hydroxyl group (OH) is introduced into an organic compound, either by directly introducing the hydroxyl group or by replacing an existing substituent with the hydroxyl group.

Furthermore, the gypenoside compound may be a stereoisomer of the compound represented by Chemical Formula 1, specifically a diastereoisomer, and more specifically a gypenoside LI compound represented by Chemical Formula 2 (gypenoside 51).

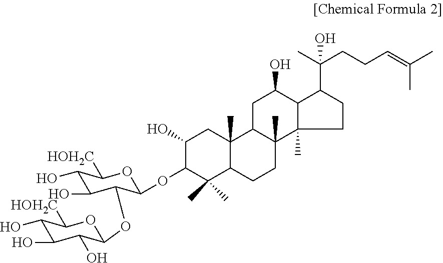

The “gypenoside LI” of the present disclosure is used interchangeably with “gypenoside LI”, “Gyp LI”, “Gyp 51”, “G51” or “gypenoside 51”, which is a diastereoisomer of the gypenoside L compound represented by Chemical Formula 1 and is a type of gypenoside (Gyp).

In a specific exemplary embodiment, the compound represented by Chemical Formula 1 or the compound represented by Chemical Formula 2 may be isolated from a Gynostemma pentaphyllum extract. For example, the Gynostemma pentaphyllum leaf extract may be an ethanol extract, a hot water extract, an ethyl acetate extract, a hexane extract or an ultra-high-pressure extract of the Gynostemma pentaphyllum leaf.

In a specific exemplary embodiment, the Gynostemma pentaphyllum leaf extract can be obtained by extracting Gynostemma pentaphyllum leaf with one or more solvent selected from a group consisting of water, a C₁₋₆ organic solvent, a subcritical fluid and a supercritical fluid. For example, it may be obtained by extracting Gynostemma pentaphyllum leaf under an ultra-high-pressure condition of 100 MPa or higher. If necessary, it can be prepared according to the methods known in the art by additionally including filtration and concentration processes. In a specific exemplary embodiment, the C₁₋₆ organic solvent may be one or more selected from a C₁₋₆ alcohol, acetone, ether, benzene, chloroform, ethyl acetate, methylene chloride, hexane, cyclohexane and petroleum ether.

Specifically, the active ingredient contained in the composition of the present disclosure may be gypenoside L, gypenoside LI or a mixture thereof.

The weight ratio of gypenoside L and gypenoside LI may be 100:20 to 80, specifically 100:30 to 70, more specifically 100:50 or 70.

When the weight of gypenoside LI for gypenoside L is within the above range, the effect of improving exercise performance can be maximized. If the weight ratio of gypenoside LI to gypenoside L is below the lower limit, the effect of improving the exercise performance of the composition becomes insignificant. And, if it exceeds the upper limit, the effect of improving exercise performance of the composition decreases.

The health functional food composition of the present disclosure may provide the desired exercise effect of improving exercise performance when it contains the gypenoside compound represented by Chemical Formula 1, a stereoisomer thereof or a sitologically acceptable salt thereof as an active ingredient in an effective amount. In this specification, the “effective amount” refers to an amount that exhibits a better response as compared to a negative control group, specifically an amount which is sufficient to improve exercise performance. The health functional food composition of the present disclosure may contain 0.001 to 99.99 wt %, specifically by 0.05 to 50 wt %, of the gypenoside compound represented by Chemical Formula 1, a stereoisomer thereof or a sitologically acceptable salt thereof, and a sitologically acceptable carrier as the balance. The effective amount of the active ingredient contained in the health functional food composition of the present disclosure will depend on the form in which the composition is produced. For the desired effect, the administration dosage of the gypenoside compound represented by Chemical Formula 1, a stereoisomer thereof or a sitologically acceptable salt thereof of the present disclosure may be 0.001 to 400 mg/kg, specifically 0.01 to 200 mg/kg, more specifically 0.01 to 100 mg/kg, more specifically 0.1 to 50 mg/kg, more specifically 1 to 20 mg/kg, more specifically 5 to 20 mg/kg, and the administration may be made 1 to 3 times a day. The administration dosage does not limit the scope of the present disclosure in any way.

The term “health functional food” used in the present disclosure refers to a food which has been prepared and processed using raw materials or ingredients with functionality useful to the human body in accordance with the Health Functional Food Act (Act No. 6727), and the “functionality” means the effect useful for the human body for health purposes such as the regulation of nutrients, physiological activities, etc.

The health functional food composition may be formulated as one selected in a group consisting of a tablet, a pill, a granule, a powder, a capsule and a liquid formulation using one or more of a carrier, a diluent, an excipient and an additive.

Furthermore, the health functional food compositions may be prepared in the form of a composition by mixing the a gypenoside compound represented by Chemical Formula 1, a stereoisomer thereof or a sitologically acceptable salt thereof with a substance or an active ingredient known to have the effect of improving exercise performance. For example, the health functional food composition of the present disclosure may further contain, in addition to the gypenoside compound, a very small amount of minerals, vitamins, sugars and other ingredients known to have the effect of improving exercise performance.

Furthermore, the present disclosure provides a pharmaceutical composition for improving exercise performance, which contains the gypenoside compound represented by Chemical Formula 1, a stereoisomer thereof or a pharmaceutically acceptable salt thereof as an active component.

The pharmaceutical composition for improving exercise performance of the present disclosure can be used for preventing or treating diseases caused by degradation of exercise performance. Examples of the diseases include degenerative diseases, mitochondrial disorders, decreased endurance, circulatory disturbance, lethargy, muscle wasting, depression, etc. The composition of the present disclosure has an effect of improving exercise performance, without limitation in the form and type of exercise. The composition of the present disclosure has an effect of improving exercise performance, without limitation in the form and type of exercise.

The pharmaceutical compositions may further contain a pharmaceutically acceptable carrier. The pharmaceutically acceptable carrier may be one commonly used for preparation, including but not limited to saline, sterile water, Ringer's solution, buffered saline, cyclodextrin, dextrose solution, maltodextrin solution, glycerol, ethanol, liposome, etc., and may contain other usual additives such as an antioxidant, a buffer, etc. as needed. Additionally, a diluent, a dispersant, a surfactant, a binder, a lubricant, etc. can be added for preparation into an aqueous solution, a suspension or an emulsion. The pharmaceutical composition of the present disclosure can be prepared as an injection, an oral formulation, a formulation for external application to skin, etc. although there is no special limitation in the formulation.

The pharmaceutical composition may be administered orally or parenterally (e.g. intravenously, subcutaneously, intraperitoneally or topically) depending on the intended method, and the administration dosage may vary depending on the patient's condition and weight, the severity of a disease, the type of a drug, and the route and time of administration, but may be appropriately selected by those skilled in the art.

In addition, the dosage level of the composition will depend on the activity of the compound, the route of administration, the severity of a condition being treated, and the condition and medical history of a patient being treated. However, the administration dosage may be increased gradually starting from an administration dosage of the compound at a lower level than required for the achievement of the desired therapeutic effect until the desired effect is achieved, within the knowledge of the related art. The desired administration dosage may be determined depending on age, sex, body type and weight. The composition may be processed further before being prepared as a pharmaceutically acceptable pharmaceutical preparation, and specifically may be crushed or ground into smaller particles. In addition, the composition will vary depending on the condition and the patient being treated, but it can be determined normally. For the desired effect, the administration dosage of the gypenoside compound represented by Chemical Formula 1, a stereoisomer thereof or a pharmaceutically acceptable salt thereof of the present disclosure may be 0.001 to 400 mg/kg, specifically 0.01 to 200 mg/kg, more specifically 0.01 to 100 mg/kg, and the administration can be made once to three times a day. The administration dosage does not limit the scope of the present disclosure in any way.

The pharmaceutical composition of the present disclosure may be prepared as a unit-dose form using a pharmaceutically acceptable carrier and/or excipient or may be packaged in a multi-dose container, according to a method that may be easily carried out by a person having common knowledge in the art to which the present disclosure belongs. Formulations in any form suitable for pharmaceutical preparations can be used, including oral formulations such as a powder, a granule, a suspension, an emulsion, a syrup, an aerosol, etc. formulations for external use such as an ointment, a cream, etc., as well as a suppository, a sterile solution for injection, and so on, and may additionally contain a dispersant or a stabilizer.

Hereinafter, the composition containing the gypenoside compound represented by Chemical Formula 1, a stereoisomer thereof or a sitologically acceptable salt thereof as an active ingredient will be described in detail through examples.

### EXAMPLES

### Preparation of Raw Materials

Gypenoside L and gypenoside LI were purchased from Embo, and gypenoside Rg3 was purchased from Sigma-Aldrich.

### Examples

Compositions of Examples 1 to 4 were prepared according to Table 1.

TABLE 1

| | Gypenoside compounds |
|---|---|
| Example 1 | Gypenoside L |
| Example 2 | Gypenoside L + gypenoside LI (100:60) |
| Example 3 | Gypenoside L + gypenoside LI (100:10) |
| Example 4 | Gypenoside L + gypenoside LI (100:100) |

### <In-Vitro Test>

### Materials and Methods

### 1. Test Substances

Creatine monohydrate (Cr) was used as a positive control substance, and all test substances were provided by BTC.

### 2. Cell Culturing and Induction of Differentiation

C2C12 cells, which are myoblasts derived from the skeletal muscle in mice, were purchased from the American Type Culture Collection (ATCC). The C2C12 cells were cultured in Dulbecco's modified Eagle's medium (DMEM) supplemented with 10% fetal bovine serum (FBS), 100 units/mL penicillin and 100 μg/mL streptomycin in a humidified CO₂ incubator (5% CO₂/95% air) at 37° C. When the cells filled about 80% of a culture dish, the single cell layer was washed with phosphate-buffered saline (PBS, pH 7.4) and the cells were detached by adding trypsin-2.65 mM EDTA and subcultured. The culture medium was replaced every two days.

When the C2C12 cells filled about 90% of the culture dish, the cells were cultured after exchanging the culture medium with a myocyte differentiation medium obtained by adding 2% horse serum (Gibco-Thermo Fisher Scientific) to DMEM in order to induce differentiation into muscle cells. The myocyte differentiation medium was replaced every two days.

3. Induction of Muscle Cell Differentiation and Treatment with Test Substances

C2C12 cells were dispensed into a 6-well plate at 2×10⁵ cells/well and stabilized for 24 hours. In order to investigate the effect of each test substance on muscle cell differentiation, each test substance was added to the myocyte differentiation medium. The cells were cultured for 4 days (mRNA analysis) or 7 days (protein analysis) after exchanging the cell culture medium with the myocyte differentiation medium treated with each test substance, as shown in Table 2.

TABLE 2

| Test groups | | Test substances | |
|---|---|---|---|
| G1 | — | — | |
| G2 | Example 1 | 0.36 | μg/mL |
| G3 | Example 2 | 0.36 | μg/mL |
| G4 | Example 3 | 0.36 | μg/mL |
| G5 | Example 4 | 0.36 | μg/mL |
| G6 | Cr | 5 | μg/mL Cr |

### 4. Measurement of Reactive Oxygen Species (ROS)

C2C12 cells were dispensed into a 96-well plate at 1×10⁴ cells/well and stabilized for 24 hours. Subsequently, the cells were cultured for 1 hour after exchanging with the culture medium with the myocyte differentiation medium containing each test substance. After 1 hour, the cells were washed with PBS and cultured further for 3 hours with a culture medium containing 50 μM tert-butyl hydrogen peroxide (TBHP), and the ROS level in the cells was measured using a DCF-DA assay kit (Abcam) according to the manufacturer's method.

### 5. Preparation of Total Cell Lysate and Investigation of Protein Expression (Western Blot Analysis)

C2C12 cells were dispensed into a 6-well plate at 2×10⁵ cells/well and stabilized for 24 hours. The cells were cultured for 7 days after exchanging the culture medium with a myocyte differentiation medium containing the test substance. After the induction of differentiation for 7 days, the cells were homogenized by adding a lysis buffer (20 mmol/L HEPES, pH 7.5, 150 mmol/L NaCl, 1% Triton X-100, 1 mmol/L EDTA, 1 mmol/L EGTA, 100 mmol/L NaF, 10 mmol/L sodium pyrophosphate, 1 mmol/L Na₃VO₄, 20 μg/mL aprotinin, 10 μg/mL antipain, 10 μg/mL leupeptin, 80 μg/mL benzamidine HCl, 0.2 mmol/L PMSF) and a total cell lysate was obtained through centrifugation. The protein quantity of the total cell lysate was measured using a BCA protein assay kit (Thermo Scientific). The total cell lysate (50 μg) was separated by 10% sodium dodecyl sulfate polyacrylamide gel electrophoresis (SDS-PAGE) and then transferred to a polyvinylidene difluoride membrane (Milipore). The membrane was blocked for 1 hour in 5% skim milk-TBST (20 mmol/L Tris·HCl, pH 7.5, 150 mmol/L NaCl, 0.1% Tween 20) and then incubated for 16 hours at 4° C. or for 1 hour at room temperature after adding antibodies. The information of the antibodies used is shown in Table 3. Thereafter, horseradish peroxidase (HRP)-linked anti-rabbit IgG or HRP-linked anti-mouse IgG was added and the mixture was stirred for 1 hour. The detected protein bands were visualized by enhanced chemiluminescence using a Luminata™ Forte Western HRP substrate (Millipore). The level of protein expression was quantified using the ImageQuant™ LAS 500 imaging system (GE Healthcare Bio-Sciences AB).

TABLE 3

| Antibodies | Details | | Manufacturers |
|---|---|---|---|
| p-AMPK | Phospho-AMPKα (Thr172) | Cat No. #2535 | Cell Signaling Technology |
| AMPK | AMPKα | Cat No. #2532 | Cell Signaling Technology |
| p-p38 | Phospho-p38 MAPK | Cat No. #9211 | Cell Signaling Technology |
| | (Thr180/Tyr182) | | |
| p38 | p38 MAPK | Cat No. #9212 | Cell Signaling Technology |
| p-Sirt1 | Phospho-Sirt1 (Ser47) | Cat No. #2314 | Cell Signaling Technology |
| Sirt1 | Sirt1 | Cat No. #9475 | Cell Signaling Technology |
| p-NRF2 | Phospho-NRF2 (Ser40) | Cat No. ab76026 | Abcam |
| NRF2 | NRF2 | Cat No. ab31163 | Abcam |
| β-Actin | Beta-actin | Cat No. #3700 | Cell Signaling Technology |

6. Investigation of mRNA Expression (Real-Time RT-PCR)

C2C12 cells were dispensed into a 6-well plate at 2×10⁵ cells/well and stabilized for 24 hours. The cells were cultured for 2 or 4 days after replacing the cell culture with a myocyte differentiation medium containing the test substance. After collecting the cells, total RNA was isolated using an RNeasy Plus Mini kit (QIAGEN), and quantified using a micro-volume spectrophotometer (BioSpec-nano, Shimadzu). RNAs with an OD260/280 value greater than 1.8 were used for experiment.

After obtaining cDNA from the total RNA (2 μg) using a HyperScript™ RT master mix kit (GeneAll Biotechnology), real-time PCR was performed using a Rotor-Gene 300 PCR kit (Corbett Research) and a Rotor-Gene™ SYBR Green kit (QIAGEN). The information of primers used in the experiment is presented in Table 4. Quantitative analysis of the gene expression was performed using the Rotor-Gene 6000 Series System Software program (Corbett Research).

TABLE 4

| | | Primer sequences | |
|---|---|---|---|
| mRNA | | (SEQ ID NO:) | Genebank No. |
| SOD2 | Forward | 5′-ATCAGGACCCATTGCAAGGA-3′ | NM_013671.3 |
| | | ((SEQ ID NO: 1) | |
| | Reverse | 5′-AGGTTTCACTTCTTGCAAGCT-3′ | NM_013671.3 |
| | | (SEQ ID NO: 2) | |
| GPx1 | Forward | 5′-CAGGTCGGACGTACTTGAG-3′ | NM_001329528.1 |
| | | (SEQ ID NO: 3) | |
| | Reverse | 5′-CAGGTCGGACGTACTTGAG-3′ | NM_001329528.1 |
| | | (SEQ ID NO: 4) | |
| UCP2 | Forward | 5′-CTCGTCTTGCCGATTGAAGGT-3′ | NM_011671.5 |
| | | (SEQ ID NO: 5) | |
| | Reverse | 5′-TCTGCAATGCAGGCAGCTGTC-3′ | NM_011671.5 |
| | | (SEQ ID NO: 6) | |
| UCP3 | Forward | 5′-GCCTACAGAACCATCGCCAG-3′ | NM_009464.3 |
| | | (SEQ ID NO: 7) | |
| | Reverse | 5′-GCCACCATCTTCAGCATACA-3′ | NM_009464.3 |
| | | (SEQ ID NO: 8) | |
| ERRα | Forward | 5′-TTCGGCGACTGCAAGCTC-3′ | NM_007953.2 |
| | | (SEQ ID NO: 9) | |
| | Reverse | 5′-CACAGCCTCAGCATCTTCAATG-3′ | NM_007953.2 |
| | | (SEQ ID NO: 10) | |
| LDB | Forward | 5′-CCTCAGATCGTCAAGTACAGCC-3′ | NM_001316322.1 |
| | | (SEQ ID NO: 11) | |
| | Reverse | 5′-ATCCGCTTCCAATCACACGGTG-3′ | NM_001316322.1 |
| | | (SEQ ID NO: 12) | |
| MCT1 | Forward | 5′-GCTGGGCAGTGGTAATTGGA-3′ | XM_021196222.2 |
| | | (SEQ ID NO: 13) | |
| | Reverse | 5′-CAGTAATTGATTTGGGAAATGCAT-3′ | XM_021196222.2 |
| | | (SEQ ID NO: 14) | |
| TFam | Forward | 5′-ATAGGCACCGTATTGCGTGA-3′ | NM_009360.4 |
| | | (SEQ ID NO: 15) | |
| | Reverse | 5′-CTGATAGACGAGGGGATGCG-3′ | NM_009360.4 |
| | | (SEQ ID NO: 16) | |
| CPT-1β | Forward | 5′-CCTGGAAGAAACGCCTGATT-3′ | NM_009948.2 |
| | | (SEQ ID NO: 17) | |
| | Reverse | 5′-CAGGGTTTGGCGAAAGAAGA-3′ | NM_009948.2 |
| | | (SEQ ID NO: 18) | |
| mtDNA | Forward | 5′-CACGATCAACTGAAGCAGCAA-3′ | NM_001362199.2 |
| | | (SEQ ID NO: 19) | |
| | Reverse | 5′-ACGATGGCCAGGAGGATAATT-3′ | NM_001362199.2 |
| | | (SEQ ID NO: 20) | |
| MHC1 | Forward | 5′-CGCTCCACGCACCCTCACTT-3′ | XM_017315841.2 |
| | | (SEQ ID NO: 21) | |
| | Reverse | 5′-GTCCATCACCCCTGGAGAC-3′ | XM_017315841.2 |
| | | (SEQ ID NO: 22) | |
| MHC7 | Forward | 5′-GCTGGAAGATGAGTGCTCAGAG-3′ | XM_017315841.2 |
| | | (SEQ ID NO: 23) | |
| | Reverse | 5′-TCCAAACCAGCCATCTCCTCTG-3′ | XM_017315841.2 |
| | | (SEQ ID NO: 24) | |
| MHC2A | Forward | 5′-CCATTCAGAGCAAAGATGCAGGG-3′ | XM_021176019.2 |
| | | (SEQ ID NO: 25) | |
| | Reverse | 5′-GCATAACGCTCTTTGAGGTTG-3′ | XM_021176019.2 |
| | | (SEQ ID NO: 26) | |
| MHC2B | Forward | 5′-GCTAGGGTGAGGGAGCTTGAA-3′ | XM_021175597.1 |
| | | (SEQ ID NO: 27) | |
| | Reverse | 5′-AGACCCTTGACGGCTTCGA-3′ | XM_021175597.1 |
| | | (SEQ ID NO: 28) | |
| PGC-1α | Forward | 5′-GTCCTTCCTCCATGCCTGAC-3′ | XM_006503779.4 |
| | | (SEQ ID NO: 29) | |
| | Reverse | 5′-GACTGCGGTTGTGTATGGGA-3′ | XM_006503779.4 |
| | | (SEQ ID NO: 30) | |
| PKB | Forward | 5′-GGACTACTTGCACTCCGAGAAG-3′ | XM_021201913.2 |
| | | (SEQ ID NO: 31) | |
| | Reverse | 5′-CATAGTGGCACCGTCCTTGATC-3′ | XM_021201913.2 |
| | | (SEQ ID NO: 32) | |
| FNDC5 | Forward | 5′-ATGAGGTGACCATGAAGGAGATGGC-3′ | XM_006503212.4 |
| | | (SEQ ID NO: 33) | |
| | Reverse | 5′-CTGGTTTCTGATGCGCTCTTGGTT-3′ | XM_006503212.4 |
| | | (SEQ ID NO: 34) | |
| GSY | Forward | 5′-CACAGAACGGTTGTCGGACTTG-3′ | NM_030678.3 |
| | | (SEQ ID NO: 35) | |
| | Reverse | 5′-AGGTGAAGTGGTCTGGAAAGGC-3′ | NM_030678.3 |
| | | (SEQ ID NO: 36) | |
| SIRT1 | Forward | 5′-GCAACAGCATCTTGCCTGAT-3′ | XM_021204930.2 |
| | | (SEQ ID NO: 37) | |
| | Reverse | 5′-GTGCTACTGGTCACTT-3′ | XM_021204930.2 |
| | | (SEQ ID NO: 38) | |
| PPARγ | Forward | 5′-CAAACACCAGTGTGAATTA-3′ | XM_021164279.2 |
| | | (SEQ ID NO: 39) | |
| | Reverse | 5′-ACCATGGTAATTTCTTGTGA-3′ | XM_021164279.2 |
| | | (SEQ ID NO: 40) | |
| GAPDH | Forward | 5′-TGGGTGTGAACCATGAGAAG-3′ | XM_029478683.1 |
| | | (SEQ ID NO: 41) | |
| | Reverse | 5′-GCTAAGCAGTTGGTGGTGC-3′ | XM_029478683.1 |
| | | (SEQ ID NO: 42) | |

### 7. Statistical Analysis

All analytical values were represented by mean±SEM. The result was analyzed using the GraphPad Prism 5.0 (GraphPad Software) program. Student's t-test and one-way analysis variance (ANOVA) were used to compare the differences between the test substance-treated groups and the control groups. Only P<0.05 was considered statistically significant.

### Test Examples

### Test Example 1: Effect on Intracellular ROS Production in C2C12 Cells

ROS are byproducts of mitochondria generated during the respiratory process of normal cells. Oxidative stress is caused by the imbalance between ROS production and antioxidant defense. Abnormally increased ROS cause dysfunction of muscle cells and lead to cell death by causing damage to macromolecules in muscle cells such as proteins, lipids, nucleic acids, etc.

In order to investigate the effect of each of the compositions according to Examples 1 to 4 on ROS production in C2C12 cells, ROS production was measured using 2′,7′-dichlorofluorescin diacetate (DCF-DA). The result is shown in Table 5. DCF-DA was measured using the principle that it is oxidized by ROS in cells and is converted to DCF, which emits fluorescence.

TABLE 5

| | Induction of | | |
|---|---|---|---|
| | oxidative | | |
| Test | stress | Test | ROS production |
| groups | (50 μM TBHP) | substances | (fluorescence intensity) |
| G0 | − | — | 16.32 ± 0.66 |
| G1 | + | — | 37.97 ± 2.19*** |
| G2 | + | Example 1 | 22.12 ± 0.70^{###} |
| G3 | + | Example 2 | 20.03 ± 0.39^{###} |
| G4 | + | Example 3 | 22.81 ± 1.15^{###} |
| G5 | + | Example 4 | 23.54 ± 2.09^{###} |
| G6 | + | Cr | 21.13 ± 2.74^{##} |

*, ** and ***indicate significant differences with p < 0.05, p < 0.01 and p < 0.001, respectively, as compared to the G0 group (for G1).
#, ^{##} and ^{###}indicate significant differences with p < 0.05, p < 0.01 and p < 0.001, respectively, as compared to the G1 group (for G2, G3, G4, G5 and G6).

As seen from Table 5, when oxidative stress was induced by treating the C2C12 cells with TBHP (G1), ROS production increased significantly to 37.97±2.19 as compared to 16.32±0.66 in the normal control group (G0). The ROS production increased by oxidative stress was significantly reduced by the treatment with the compositions of Examples 1 to 5. The treatment with STO slightly reduced ROS production to 33.97±2.22 as compared to the oxidative stress-induced group (G1). When the positive control substance, Cr, was treated, ROS production was significantly reduced to 21.13±2.74 as compared to the oxidative stress-induced control group (G1). This indicates that the gypenoside compounds effectively inhibit the production of ROS owing to oxidative stress induced by TBHP in the C2C12 cells.

### Test Examples 2: Effect on Change in Proteins Related with Peroxisome Proliferator-Activated Receptor Gamma Coactivator-1 Alpha (PGC-1α) in C2C12 Cells

Mitochondria are powerhouses in muscle, which oxidize energy sources and produce ATP. When high exercise load is given continuously, the number and quantity of mitochondria are increased for more oxidation of energy sources. PGC-1α regulates mitochondrial function, biosynthesis and cellular energy metabolism. The activation of PGC-1α is induced by AMP-activated protein kinase (AMPK) and silent mating type information regulation 2 homolog 1 (Sirt1). It has been reported that its activation is increased by endurance exercise. AMPK is an enzyme that detects the energy state within the cell and regulates various metabolic pathways to restore normal energy balance when energy is lacking in the cell, i.e., when AMP is increased as compared to ATP. Sirt1 is recognized as an important regulator for mitochondrial biosynthesis in skeletal muscle through exercise, because its activity is increased by the change in NAD⁺ following muscle contraction and the activity of PGC-1α is regulated. p38 mitogen-activated protein kinase (MAPK) is an enzyme known to be activated by various extracellular stimulations and involved in cell growth and differentiation, cell cycle regulation, and so on. Exercise or the contraction of skeletal muscle increases the activity of p38 MAPK, and it has been recently reported that p38 MAP activates PGC-1α.

The result of western blot analysis conducted using the total cell lysate obtained by treating with the test substance to investigate the effect of the test substance on the changes in proteins associated with PGC-1α activation is shown in Table 6. Table 6 shows the expression level of the proteins associated with PGC-1α activation for each test group determined by comparing relative band densities (% of control group).

TABLE 6

| Test | Test | Protein expression level (% of control group) | | | |
|---|---|---|---|---|---|
| groups | substances | p-AMPK | p-Sirt1 | p-p38 | p-Nrf2 |
| G1 | — | 100 ± 0.0 | 100 ± 0.0 | 100 ± 0.0 | 100 ± 0.0 |
| G2 | Example 1 | 117.7 ± 7.2 | 143.4 ± 5.6* | 133.1 ± 7.1* | 135.1 ± 1.6*** |
| G3 | Example 2 | 135.8 ± 6.2 | 137.4 ± 4.4 | 140.8 ± 11.6 | 146.5 ± 3.4 |
| G4 | Example 3 | 119.6 ± 3.4 | 142.1 ± 2.8 | 135.2 ± 6.4 | 134.7 ± 2.5 |
| G5 | Example 4 | 120.4 ± 5.9 | 141.9 ± 5.7 | 134.1 ± 5.2 | 133.6 ± 3.6 |
| G6 | Cr | 104.6 ± 14.4 | 114.0 ± 11.1 | 151.5 ± 41.0 | 116.2 ± 11.4 |

Based on the western blot analysis result, the activity of each protein was evaluated from the ratio of activation. The result is shown in Table 7.

TABLE 7

| | | p-AMPK/ | p-Sirt1/ | p-p38/ | p-Nrf2/ |
|---|---|---|---|---|---|
| | | AMPK | Sirt1 | p38 | Nrf2 |
| Test groups | | ratio | ratio | ratio | ratio |
| G1 | — | 1.00 ± 0.00 | 1.00 ± 0.00 | 1.00 ± 0.00 | 1.00 ± 0.00 |
| G2 | Example 1 | 1.27 ± 0.25 | 1.38 ± 0.06* | 1.27 ± 0.07* | 1.50 ± 0.11* |
| G3 | Example 2 | 1.52 ± 0.14 | 1.32 ± 0.03* | 1.38 ± 0.10* | 1.61 ± 0.10* |
| G4 | Example 3 | 1.28 ± 0.29 | 1.37 ± 0.07* | 1.28 ± 0.10* | 1.51 ± 0.12* |
| G5 | Example 4 | 1.29 ± 0.07 | 0.38 ± 0.11* | 1.27 ± 0.16* | 1.50 ± 0.09* |
| G6 | Cr | 1.09 ± 0.13 | 1.11 ± 0.10 | 1.15 ± 0.29 | 1.20 ± 0.26 |

*, ** and ***indicate significant differences with p < 0.05, p < 0.01 and p < 0.001, respectively, as compared to the G1 group (G2 to G6).

As seen from Table 6 and Table 7, the expression of p-AMPK was increased by the treatment of the compositions of the examples (G2 to G5) as compared to the control group (G1). The expression of p-Sirt1 was significantly increased by the treatment of the compositions of the examples (G2 to G5) as compared to the control group (G1). The expression of p-p38 MAPK was significantly increased by the treatment of the compositions of the examples (G2 to G5). The p-p38/p38 ratio was significantly increased by the treatment of the compositions of the examples.

Activated PGC-1α induces the activation of various other transcription factors including nuclear factor erythroid-2 related factor 2 (Nrf2), which is a leucine zipper transcription factor, and thereby regulates the expression of antioxidant genes. When oxidative stress is induced, Nrf2 migrates from the cytoplasm to the nucleus and binds to the promoter moiety of antioxidant genes to induce the expression of various antioxidant genes. The expression of p-Nrf2 was significantly increased by the treatment of the compositions of the examples. The p-Nrf2/Nrf2 ratio was also increased by the treatment of the compositions of the examples.

The treatment with the compositions of the examples significantly increased the activation of Sirt1, p38 MAPK and Nrf2 in the C2C12 cells. On the other hand, the treatment with STO, which is an inhibitor of CaMKK, significantly inhibited the activation of AMPK, p38 MAPK and Nrf2, suggesting that they are activated in response to change in the Ca²⁺ level. The inhibited activation of AMPK, p38 MAPK and Nrf2 tended to be recovered by the compositions of the examples, but there was no significant difference.

### Test Example 3: Effect on mRNA Expression Related with Mitochondrial Replication in C2C12 Cells

Mitochondria are essential organelles for life because they are central to many cellular processes, including ATP production, cell death, beta oxidation of fatty acids, synthesis of iron-sulfur clusters, etc. Mitochondria have their own unique genome in the form of mitochondrial DNA (mtDNA), which is different from that of the chromosomal DNA present in the nucleus.

Mitochondrial transcription factor A (Tfam) transforms the nucleic structure of the mitochondria to protect the DNA from the attack of ROS and, thereby, regulates the stability and transcription of the mtDNA. Carnitine palmitoyl transferase-1 (CPT-1) is associated with fat oxidation among the genetic characteristics associated with ADP phosphorylation within the mitochondria. It is an enzyme involved in the inflow of long-chain fatty acyl-CoA that has passed through the mitochondrial outer membrane into the mitochondrial matrix via the inner membrane.

The effect of the treatment of C2C12 cells with the test substances on the mRNA expression of mtDNA, Tfam and CPT-1β associated with mitochondrial replication was investigated. The result is shown in Table 8.

TABLE 8

| Test groups | | TFam | CPT1-β | mtDNA |
|---|---|---|---|---|
| G1 | — | 1.00 ± 0.00 | 1.00 ± 0.00 | 1.00 ± 0.00 |
| G2 | Example 1 | 0.91 ± 0.10 | 1.83 ± 0.41 | 1.29 ± 0.41 |
| G3 | Example 2 | 0.98 ± 0.23 | 2.53 ± 0.61 | 1.07 ± 0.29 |
| G4 | Example 3 | 0.92 ± 0.12 | 1.81 ± 0.17 | 1.27 ± 0.13 |
| G5 | Example 4 | 0.92 ± 0.06 | 1.80 ± 0.23 | 1.29 ± 0.36 |
| G6 | Cr | 0.48 ± 0.07** | 0.93 ± 0.20 | 0.72 ± 0.12* |

*, ** and ***indicate significant differences with p < 0.05, p < 0.01 and p < 0.001, respectively, as compared to the G1 group (G2 to G6).

As shown in Table 8, there was no significant difference in all the groups in the mRNA expression of Tfam. The mRNA expression of CPT1-β showed a tendency to increase in the groups treated with the compositions of the examples (G2 to G5). The mRNA expression of mtDNA decreased significantly in the Cr-treated group (G6) as compared to the control group (G1).

### Test Example 4: Effect on mRNA Expression of Genes Related with Change in Muscular Strength Type in C2C12 Cells

The effect of the compositions according to Examples 1 to 4 on the mRNA expression of MHC1, MHC7, MHC2A and MHC2B, which are responsible for muscular strength type, was investigated. The result is shown in Table 9.

TABLE 9

| Test groups | | MHC1 | MHC7 | MHC2A | MHC2B |
|---|---|---|---|---|---|
| G1 | — | 1.00 ± 0.00 | 1.00 ± 0.00 | 1.00 ± 0.00 | 1.00 ± 0.00 |
| G2 | Example 1 | 10.30 ± 1.75** | 1.80 ± 0.41 | 1.54 ± 0.32 | 1.20 ± 0.12 |
| G3 | Example 2 | 12.93 ± 1.47** | 2.53 ± 0.27 | 1.77 ± 0.27 | 1.51 ± 0.12 |
| G4 | Example 3 | 8.55 ± 1.64** | 1.79 ± 0.32 | 1.37 ± 0.24 | 1.12 ± 0.16 |
| G5 | Example 4 | 9.61 ± 0.95** | 1.94 ± 0.23 | 1.44 ± 0.18 | 1.19 ± 0.21 |
| G6 | Cr | 4.50 ± 1.43 | 7.87 ± 2.66* | 1.74 ± 0.22* | 1.10 ± 0.10 |

*, ** and ***indicate significant differences with p < 0.05, p < 0.01 and p < 0.001, respectively, as compared to the G1 group (G2 to G6).

Myosin is the most abundant protein in skeletal muscles and is involved in muscle contraction. Myosin consists of myosin heavy chains (MHC) and myosin light chains (MLC). The MHC is an important factor that determines the type of muscle contraction. Among the well-known subtypes in skeletal muscle, MHC2A and MHC2B subtypes are known to be mainly involved in fast muscle contraction, and MHC1 and MHC7 subtypes are known to be involved in slow muscle contraction. The mRNA expression of MHC1 increased significantly as compared to the control group (G1) in the groups treated with the compositions of the examples (G2 to G5). The mRNA expression of MHC2A tended to increase in the groups treated with the compositions of the examples (G2 to G5), but there was no significant difference. That is to say, it is thought that the treatment with the compositions of the examples induces differentiation into the slow muscle type involved in slow muscle contraction by increasing the mRNA expression of MHC1 and MHC7.

The result of investigating the effect of the compositions according to Examples 1 to 4 on the mRNA expression of PGC-1α, PKB and FNDC5 on the muscular strength type is shown in Table 10.

TABLE 10

| Test groups | | PGC-1α | PKB | FNDC5 |
|---|---|---|---|---|
| G1 | — | 1.00 ± 0.00 | 1.00 ± 0.00 | 1.00 ± 0.00 |
| G2 | Example 1 | 11.75 ± 3.82* | 3.04 ± 0.46** | 2.97 ± 0.39** |
| G3 | Example 2 | 13.23 ± 2.64* | 4.13 ± 0.28** | 3.31 ± 0.38** |
| G4 | Example 3 | 11.54 ± 2.13* | 3.11 ± 0.45** | 2.84 ± 0.24** |
| G5 | Example 4 | 11.86 ± 1.61* | 3.21 ± 0.60** | 2.89 ± 0.33** |
| G6 | Cr | 1.54 ± 1.10 | 2.18 ± 0.51 | 1.83 ± 0.47 |

*, ** and ***indicate significant differences with p < 0.05, p < 0.01 and p < 0.001, respectively, as compared to the G1 group (G2 to G6).

PGC-1α is known as a transcriptional coactivator that plays a key role in regulation of genes for skeletal muscle adaptation to exercise such as mitochondrial biosynthesis, fast-to-slow fiber type switching, etc. The mRNA expression of PGC-1α increased significantly as compared to the control group (G1) in the groups treated with the compositions of the examples (G2 to G5). Protein kinase B (PKB), also known as Akt, plays an important role in glucose metabolism and several cellular metabolism processes. The PKB, which is an upstream signal transduction factor of GLUT4, transmits signals to GLUT4 and, thereby, allows glucose to be transported in the muscle. The mRNA expression of PKB increased significantly as compared to the control group (G1) in the groups treated with the compositions of the examples (G2 to G5). PGC-1α, which is activated in skeletal muscle in response to exercise, is involved in the oxidative stress control mechanism together with FNDC5 (fibronectin type III domain-containing protein 5), which is a skeletal membrane protein and also activates the insulin signal transduction pathway to enhance insulin sensitivity. The mRNA expression of FNDC5 increased significantly in the groups treated with the compositions of the examples (G2 to G5) as compared to the control group (G1), and no significant difference was observed in all other groups.

### Test Example 5: Effect on mRNA Expression of Genes Related with Energy in C2C12 Cells

The result of investigating the effect of the compositions according to Examples 1 to 4 on the mRNA expression of GSY, SIRT1 and PPARγ involved in the energy metabolism of muscle cells is shown in Table 11.

TABLE 11

| Test groups | | GSY | Sirt1 | PPARγ |
|---|---|---|---|---|
| G1 | — | 1.00 ± 0.00 | 1.00 ± 0.00 | 1.00 ± 0.00 |
| G2 | Example 1 | 2.71 ± 0.32** | 1.40 ± 0.22 | 1.16 ± 0.47 |
| G3 | Example 2 | 3.06 ± 0.29** | 1.64 ± 0.17 | 1.29 ± 0.34 |
| G4 | Example 3 | 2.64 ± 0.31** | 1.36 ± 0.09 | 1.10 ± 0.11 |
| G5 | Example 4 | 2.70 ± 0.18** | 1.38 ± 0.18 | 1.18 ± 0.20 |
| G6 | Cr | 2.30 ± 0.39* | 1.13 ± 0.18 | 2.93 ± 1.32 |

*, ** and ***indicate significant differences with p < 0.05, p < 0.01 and p < 0.001, respectively, as compared to the G1 group (G2 to G6).

Glycogen synthase (GSY) is a key enzyme of glycogenesis involved in the synthesis and storage of glycogen by insulin. Glycogen in skeletal muscle is a major source of energy during exercise. The higher the intensity of exercise, the greater the energy dependence on glycogen. The mRNA expression of GSY increased significantly as compared to the control group (G1) in the groups treated with the compositions of the examples (G2 to G5) and also increased significantly in the group treated with the positive control substance Cr (G6). Energy requirement increases rapidly as the amount of ATP stored in skeletal muscle decreases during exercise. The associated increase in NAD⁺ within cells activates Sirt1 and the activated Sirt1 expresses transcription factors in the cytoplasm and nucleus for generation of energy necessary for exercise. The mRNA expression of Sirt1 showed a tendency to increase in the groups treated with the compositions of the examples (G2 to G5) as compared to the control group (G1), but there was no significant difference. Peroxisome proliferator-activated receptor gamma (PPAR-γ) is a transcription factor and a member of nuclear receptors. It is activated by a ligand, regulates genes associated with lipid and glucose metabolism and energy homeostasis, and regulates cellular proliferation and differentiation.

In addition, PPAR-γ regulates the ability to oxidize fatty acids in skeletal muscle through interaction with PGC-1α. The mRNA expression of PPAR-γ tended to increase as compared to the control group (G1) in the groups treated with the compositions of the examples (G2 to G5), but no significant difference was observed.

Through the above results, it was confirmed that the gypenoside compounds, which are the active ingredients of the compositions of the examples, suppress oxidative stress and increase the expression of various genes associated with mitochondrial biosynthesis in muscle cells. Therefore, it is thought that the compositions of the examples can be used as functional substances for alleviating fatigue caused by exercise and improving exercise performance.

### <In-Vivo Test>

### Materials and Methods

### 1. Test Substances

Creatine monohydrate (Cr) was used as a positive control substance.

### 2. Approval of Animal Experiments

All animal experiments were carried out under the approval of the Institutional Animal Care and Use Committee of Hallym University (Hallym 2019-28).

3. Evaluation of Muscle Fatigue-Alleviating Effect of Gypenoside in In-Vivo System—without Exercise

### (1) Experimental Animals

Specific-pathogen-free 5-week-old male ICR mice were purchased from DooYeol Biotech. After a week of quarantine and acclimatization, healthy animals without weight loss were selected and used for experiment. The animals were raised in breeding environments maintained at 23±3° C. with relative humidity of 50±10%, ventilation with 10 to 15 times/hour, lighting for 12 hours (08:00 to 20:00), and light intensity of 150 to 300 Lux. Throughout the test period, the experimental animals were given free access to solid feed for experimental animals (Cargill Agri Puna) and drinking water.

TABLE 12

| Test groups | | Number of animals | Test substances (mg/kg BW) |
|---|---|---|---|
| G1 | — | 10 | — |
| G2 | Example 1 | 10 | 7 |
| G3 | Example 2 | 10 | 7 |
| G4 | Example 3 | 10 | 7 |
| G5 | Example 4 | 10 | 7 |

### (2) Test Groups and Administration of Test Substances

After an acclimatization period of 1 week, healthy animals were selected and divided into 6 groups of 10 mice per group according to the randomized block design. A normal control group was orally administered with 5% Tween 80-saline, and a composition was orally administered with Creatine monohydrate (Cr) at 75 mg/kg body weight (BW). Test groups (G2 to G5) were orally administered the compositions of Examples 1 to 4 dissolved in drinking water to 7 mg/kg body weight (BW) at regular times for 17 days. Throughout the test period, the experimental animals were given free access to solid feed for experimental animals (Cargill Agri Puna) and drinking water.

### (3) Measurement of Body Weight

The body weight of the experimental animals was measured every week at regular times during the test period.

### (4) Forced Swimming Test and Measurement of Blood Lactate Content

Weight-loaded forced swimming test was performed for the experimental animals as follows. A plastic pool (90×45×45 cm) was filled with water at the depth of 35 cm and the temperature of the water was maintained at 25±1° C. After attaching a weight (5% of the body weight of the experimental animal) to the tail, the experimental animal was forced to swim in the pool. The experimental animal was judged to be exhausted if it did not rise to the surface of water for 7 seconds.

On days 10 and 12 after the administration of the test substance, swimming adaptation exercise was given twice for 15 minutes each. Two days after the final swimming adaptation exercise (on day 14 after the administration of the test substance), forced swim test was performed after fasting the experimental animal for 16 hour, and the time until exhaustion was measured.

The experimental animal's blood lactate content was measured using a lactate measurement device (Lactate Pro2, Arkray) before, immediately after, and 10 minutes and 30 minutes after the forced swimming.

### (5) Blood Sampling

On day 17 after the administration of the test substance, the experimental animal was forced to swim (for 60 minutes) without a weight load. Then, the experimental animal was anesthetized using an anesthetic prepared by dilution of tribromoethanol with tertiary amyl alcohol and blood was taken from the eye socket. The blood was placed in a serum-separating tube (Becton Dickinson) and left at room temperature for 30 minutes, centrifuged at 3,000 rpm for 20 minutes to separate the serum, and kept at −70° C. until analysis.

### (6) Serum Analysis

Blood urea nitrogen (BUN) and creatinine (CREA) content in the serum and the activities of alanine aminotransferase (ALT), aspartate aminotransferase (AST), creatine kinase (CK) and lactate dehydrogenase (LDH) were measured using a blood biochemistry analyzer (KoneLab 20 XT, Thermo Fisher Scientific). The serum lactate content was measured using a lactate assay kit (Abcam) according to the method proposed by the manufacturer.

4. Evaluation of Exercise Performance-Enhancing Effect of Gypenoside in In-Vivo System—with Exercise

### (1) Experimental Animals

Specific-pathogen-free 5-week-old male ICR mice were purchased from DooYeol Biotech. After a week of quarantine and acclimatization, healthy animals without weight loss were selected and used for experiment. The animals were raised in breeding environments maintained at 23±3° C. with relative humidity of 50±10%, ventilation with 10 to 15 times/hour, lighting for 12 hours (08:00 to 20:00), and light intensity of 150 to 300 Lux. Throughout the test period, the experimental animals were given free access to solid feed for experimental animals (Cargill Agri Puna) and drinking water.

### (2) Test Groups and Administration of Test Substances

After an acclimatization period of 1 week, healthy animals were selected and divided into 6 groups according to the randomized block design. The animals were divided into (G1) a non-exercise control group, (G2) a non-exercise+7 mg/kg body weight (BW) Example 1 group, (G3) a non-exercise+7 mg/kg BW Example 2 group, (G4) a non-exercise+7 mg/kg BW Example 3 group, (G5) a non-exercise+7 mg/kg BW Example 4 group, (G6) an exercise control group, (G7) an exercise+7 mg/kg BW Example 1 group, (G8) an exercise+7 mg/kg BW Example 2 group, (G9) an exercise+7 mg/kg BW Example 3 group, (G10) an exercise+7 mg/kg BW Example 4 group, and (G11) an exercise+75 mg/kg BW Cr dose group. Each test group consisted of 10 animals.

The test substance was dissolved in drinking water and administered every day at regular times (2 hours before exercise) for 6 weeks. Throughout the test period, the experimental animals were given free access to solid feed for experimental animals (Cargill Agri Puna) and drinking water.

TABLE 13

| | Number of | Treadmill | Test substances |
|---|---|---|---|
| Test groups | animals | exercise | (mg/kg BW) |

| | | | | |
|---|---|---|---|---|
| G1 | — | 10 | − | — |
| G2 | Example 1 | 10 | − | 7 |
| G3 | Example 2 | 10 | − | 7 |
| G4 | Example 3 | 10 | − | 7 |
| G5 | Example 4 | 10 | − | 7 |
| G6 | — | 10 | + | — |
| G7 | Example 1 | 10 | + | 7 |
| G8 | Example 2 | 10 | + | 7 |
| G9 | Example 3 | 10 | + | 7 |
| G10 | Example 4 | 10 | + | 7 |
| G11 | Cr | 10 | + | 75 |

### (3) Measurement of Body Weight and Feed Intake

The body weight of the experimental animals was measured every week at regular times during the test period. The feed intake of the experimental animals was measured throughout the test period. Total feed intake and daily feed intake were recorded.

### (4) Treadmill Test and Endurance Exercise

The endurance exercise of the animals was conducted for 6 weeks using a treadmill for small animals (Exer3/6 Treadmill, Columbus Instruments). The endurance exercise was conducted with 10 degrees of slope at 10 m/min, starting from for 15 minutes in the first week, for 20 minutes in the second week, for 25 minutes in the third week, for 30 minutes in the fourth week, for 35 minutes in the fifth week, and for 40 minutes in the sixth week.

After 6 weeks of the exercise training, in order to evaluate endurance exercise performance, exercise was started with 10 degrees of slope at 10 m/min for 5 minutes, and the intensity of the exercise was increased by 1 m/min every 1 minute up to the highest speed of 25 m/min. The duration of the exercise until exhaustion was measured. The experimental animal was judged to be exhausted if it was unable to run for 10 seconds or longer at the back of the treadmill. The exercise capacity of the experimental animal was calculated as follows.

$$Exercise capacity = body weight ( kg ) \times speed ( m / s ) \times time ( s ) \times 9.8 m / {s}^{2}$$

### (5) Measurement of Lean Body Mass and Body Fat Percentage

One day before the end of the test, the experimental animal was anesthetized and the lean body mass and body fat percentage were measured by dual-energy X-ray absorptiometry (DEXA, PIXImus™, GE Lunar).

### (6) Blood Sampling and Tissue Extraction

Before sacrificing, the experimental animal was anesthetized using an anesthetic prepared by dilution of tribromoethanol with tertiary amyl alcohol and blood was taken from the eye socket. The blood was placed in a serum-separating tube (Becton Dickinson) and left at room temperature for 30 minutes, centrifuged at 5,000 rpm for 10 minutes to separate the serum, and kept at −70° C. until analysis. After the blood sampling, the experimental animal was sacrificed and the liver and skeletal muscles [quadriceps femoris muscle (QF), gastrocnemius muscle (GA), soleus muscle (SOL), and extensor digitorum longus muscle (EDL)] were extracted. Their weight was measured after rinsing with cold physiological saline and removing excess water with filter paper. A portion of the soleus muscle (SOL) was fixed with 4% paraformaldehyde (PFA) and then embedded in paraffin for histoimmunological staining. Also, total RNA was isolated from a portion of the soleus muscle and subjected to real-time RT-PCR. Proteins were isolated from a portion of the gastrocnemius muscle (GA) and subjected to western blot. The remaining tissue was kept at −70° C. until analysis.

### (7) Blood Biochemistry Analysis

The contents of glucose, triglycerides (TG), total cholesterol (CHOL), LDL-cholesterol (LDL-CHOL), HDL-cholesterol (HDL-CHOL), blood urea nitrogen (BUN) and creatinine (CREA) in the serum, and the activities of creatine kinase (CK), lactate dehydrogenase (LDH), alanine aminotransferase (ALT), aspartate aminotransferase (AST) and alkaline phosphatase (ALP) were measured using a blood biochemistry analyzer (KoneLab 20 XT, Thermo Fisher Scientific). The lactate content in the serum was measured using a lactate measurement kit (Abcam) according to the method proposed by the manufacturer.

### (8) Preparation of Liver Tissue Fluid

A liver tissue fluid was prepared to measure glycogen content in the liver tissue. 100 mg of liver tissue was homogenized with a homogenizer after adding 1 mL of PBS. The homogenized solution was centrifuged at 5,000 rpm for 10 minutes and then used as a liver tissue fluid.

### (9) Preparation of Muscle Tissue Fluid

A muscle tissue fluid was prepared to measure glycogen content and enzymatic activity in the muscle tissue. 1 mL of PBS was added to the extracted quadriceps femoris muscle (QF), gastrocnemius muscle (GA), soleus muscle (SOL) and extensor digitorum longus muscle (EDL), and the skeletal muscles were homogenized with a homogenizer. The homogenized solution was centrifuged at 5,000 rpm for 10 minutes and then used as a muscle tissue fluid. The amount of proteins in the muscle tissue was measured using a BCA protein assay kit (Thermo Scientific).

### (10) Measurement of Glycogen Content in Liver and Skeletal Muscle

The glycogen content in the liver and skeletal muscle (GA) was measured using a glycogen assay kit according to the method presented by the manufacturer (Abcam).

### (11) Measurement of Enzymatic Activity in Skeletal Muscles

The activity of citrate synthase (BioVision), beta-hydroxyacyl CoA-dehydrogenase (MyBioSource) and lactate dehydrogenase (Abcam) in the skeletal muscles (QF, SOL and EDL) was measured using respective assay kits according to the methods presented by the manufacturers.

### (12) Histomorphological Observation of Skeletal Muscle (Hematoxylin & Eosin Staining)

The soleus muscle fixed with 4% PFA was embedded in paraffin and 5-μm tissue slices were prepared from the embedded tissue. After the removal of paraffin, the tissue was hydrated by gradually lowering the percentage of alcohol from 100% alcohol to 0% alcohol (H₂O). For histomorphological observation of the soleus muscle (SOL), the tissue was stained with Accustain® hematoxylin and eosin stains (Sigma-Aldrich Co.) in accordance with the manufacturer's method. Subsequently, the histological change was observed using an optical microscope (Carl Zeiss).

### (13) Investigation of Protein Expression in Muscle Tissue (Western Blot Analysis)

For investigation of protein expression, the muscle tissue (gastrocnemius muscle, GA) was homogenized with a homogenizer after adding a lysis buffer (20 mmol/L HEPES, pH 7.5, 150 mmol/L NaCl, 1% Triton X-100, 1 mmol/L EDTA, 1 mmol/L EGTA, 100 mmol/L NaF, 10 mmol/L sodium pyrophosphate, 1 mmol/L Na₃VO₄, 20 μg/mL aprotinin, 10 μg/mL antipain, 10 μg/mL leupeptin, 80 μg/mL benzamidine HCl, 0.2 mmol/L PMSF). The homogenized solution was centrifuged for 10 minutes at 12,000 rpm and the supernatant was taken to obtain a muscle tissue lysate. The amount of proteins in the muscle tissue lysate was measured using a BCA protein assay kit (Thermo Scientific).

The proteins (50 μg) were separated by 10% sodium dodecyl sulfate polyacrylamide gel electrophoresis (SDS-PAGE) and then transferred to a polyvinylidene difluoride membrane (Milipore). The membrane was blocked for 1 hour in 5% skim milk-TBST (20 mmol/L Tris-HCl, pH 7.5, 150 mmol/L NaCl, 0.1% Tween 20) and then stirred for 16 hours at 4° C. or for 1 hour at room temperature after adding antibodies. The information of the antibodies used is shown in Table 14. Thereafter, horseradish peroxidase (HRP)-linked anti-rabbit IgG or HRP-linked anti-mouse IgG was added and the mixture was stirred for 1 hour. The detected protein bands were visualized by enhanced chemiluminescence using a Luminata™ Forte Western HRP substrate (Millipore). The expression level of the proteins was quantified using an ImageQuant™ LAS 500 imaging system (GE Healthcare Bio-Sciences AB).

TABLE 14

| Antibodies | Details | | Manufacturers |
|---|---|---|---|
| p-AMPK | Phospho-AMPKα | Cat No. #2535 | Cell Signaling |
| | (Thr172) | | Technology |
| AMPK | AMPKα | Cat No. #2532 | Cell Signaling |
| | | | Technology |
| p-p38 | Phospho-p38 MAPK | Cat No. #9211 | Cell Signaling |
| | (Thr180/Tyr182) | | Technology |
| p38 | p38 MAPK | Cat No. #9212 | Cell Signaling |
| | | | Technology |
| p-PGC1α | Phospho-PGC1α | Cat No. AF6650 | R&D Systems |
| | (Ser571) | | |
| PGC1α | PGC1α | Cat No. #4259 | Cell Signaling |
| | | | Technology |
| p-NRF2 | Phospho-NRF2 | Cat No. ab76026 | Abcam |
| | (Ser40) | | |
| NRF2 | NRF2 | Cat No. ab31163 | Abcam |
| β-Actin | Beta-actin | Cat No. #3700 | Cell Signaling |
| | | | Technology |

(14) Investigation of mRNA Expression in Muscle Tissue (Real-Time RT-PCR)

Total RNA was isolated from the muscle tissue (SOL) collected at the end of the test using a TRizol reagent (Thermo Fisher Scientific), and quantified using a micro-volume spectrophotometer (BioSpec-nano, Shimadzu). RNAs with OD260/280 values above 1.8 were used for experiment. After obtaining cDNA from the total RNA (2 μg) using a HyperScript™ RT master mix kit (GeneAll Biotechnology), real-time PCR was performed using a Rotor-Gene 300 PCR kit (Corbett Research) and a Rotor-Gene™ SYBR Green kit (QIAGEN). The information of primers used in the experiment is presented in Table 15. Quantitative analysis of the gene expression was performed using the Rotor-Gene 6000 Series System Software program (Corbett Research).

TABLE 15

| | | Primer sequences | |
|---|---|---|---|
| mRNA | | (SEQ ID NO: ) | Genebank No. |
| SOD2 | Forward | 5′-ATCAGGACCCATTGCAAGGA-3′ | NM_013671.3 |
| | | (SEQ ID NO: 1) | |
| | Reverse | 5′-AGGTTTCACTTCTTGCAAGCT-3′ | NM_013671.3 |
| | | (SEQ ID NO: 2) | |
| GPx1 | Forward | 5′-CAGGTCGGACGTACTTGAG-3′ | NM_001329528.1 |
| | | (SEQ ID NO: 3) | |
| | Reverse | 5′-CAGGTCGGACGTACTTGAG-3′ | NM_001329528.1 |
| | | (SEQ ID NO: 4) | |
| UCP2 | Forward | 5′-CTCGTCTTGCCGATTGAAGGT-3′ | NM_011671.5 |
| | | (SEQ ID NO: 5) | |
| | Reverse | 5′-TCTGCAATGCAGGCAGCTGTC-3′ | NM_011671.5 |
| | | (SEQ ID NO: 6) | |
| UCP3 | Forward | 5′-GCCTACAGAACCATCGCCAG-3′ | NM_009464.3 |
| | | (SEQ ID NO: 7) | |
| | Reverse | 5′-GCCACCATCTTCAGCATACA-3′ | NM_009464.3 |
| | | (SEQ ID NO: 8) | |
| PGC1α | Forward | 5′-GTCCTTCCTCCATGCCTGAC-3′ | XM_006503779.4 |
| | | (SEQ ID NO: 29) | |
| | Reverse | 5′-GACTGCGGTTGTGTATGGGA-3′ | XM_006503779.4 |
| | | (SEQ ID NO: 30) | |
| PPARδ | Forward | 5′-GGACCAGAACACACGCTTCCTT-3′ | NM_011145.3 |
| | | (SEQ ID NO: 45) | |
| | Reverse | 5′-CCGACATTCCATGTTGAGGCTG-3′ | NM_011145.3 |
| | | (SEQ ID NO: 46) | |
| MCT1 | Forward | 5′-GCTGGGCAGTGGTAATTGGA-3′ | XM_021196222.2 |
| | | (SEQ ID NO: 13) | |
| | Reverse | 5′-CAGTAATTGATTTGGGAAATGCAT-3′ | XM_021196222.2 |
| | | (SEQ ID NO: 14) | |
| CPT-1β | Forward | 5′-CCTGGAAGAAACGCCTGATT-3′ | NM_009948.2 |
| | | (SEQ ID NO: 17) | |
| | Reverse | 5′-CAGGGTTTGGCGAAAGAAGA-3′ | NM_009948.2 |
| | | (SEQ ID NO: 18) | |
| ERRα | Forward | 5′-TTCGGCGACTGCAAGCTC-3′ | NM_007953.2 |
| | | (SEQ ID NO: 9) | |
| | Reverse | 5′-CACAGCCTCAGCATCTTCAATG-3′ | NM_007953.2 |
| | | (SEQ ID NO: 10) | |
| LDH B | Forward | 5′-CCTCAGATCGTCAAGTACAGCC-3′ | NM_001316322.1 |
| | | (SEQ ID NO: 11) | |
| | Reverse | 5′-ATCCGCTTCCAATCACACGGTG-3′ | NM_001316322.1 |
| | | (SEQ ID NO: 12) | |
| mtDNA | Forward | 5′-CACGATCAACTGAAGCAGCAA-3′ | NM_001362199.2 |
| | | (SEQ ID NO: 19) | |
| | Reverse | 5′-ACGATGGCCAGGAGGATAATT-3′ | NM_001362199.2 |
| | | (SEQ ID NO: 20) | |
| NRF-1 | Forward | 5′-GGCAACAGTAGCCACATTGGCT-3′ | XM_030255219.1 |
| | | (SEQ ID NO: 43) | |
| | Reverse | 5′-GTCTGGATGGTCATTTCACCGC-3′ | XM_030255219.1 |
| | | (SEQ ID NO: 44) | |
| Tfam | Forward | 5′-ATAGGCACCGTATTGCGTGA-3′ | NM_009360.4 |
| | | (SEQ ID NO: 15) | |
| | Reverse | 5′-CTGATAGACGAGGGGATGCG-3′ | NM_009360.4 |
| | | (SEQ ID NO: 16) | |
| IL-6 | Forward | 5′-CCTCTGGTCTTCTGGAGTACC-3′ | NM_031168.2 |
| | | (SEQ ID NO: 47) | |
| | Reverse | 5′-ACTCCTTCTGTGACTCCAGC-3′ | NM_031168.2 |
| | | (SEQ ID NO: 48) | |
| TNF-α | Forward | 5′-ATGAGCACAGAAAGCATGA-3′ | XM_021149735.1 |
| | | (SEQ ID NO: 49) | |
| | Reverse | 5′-AGTAGACAGAAGAGCGTGGT-3′ | XM_021149735.1 |
| | | (SEQ ID NO: 50) | |
| PPARγ | Forward | 5′-CAAACACCAGTGTGAATTA-3′ | XM_021164279.2 |
| | | (SEQ ID NO: 39) | |
| | Reverse | 5′-ACCATGGTAATTTCTTGTGA-3′ | XM_021164279.2 |
| | | (SEQ ID NO: 40) | |
| GSY | Forward | 5′-CACAGAACGGTTGTCGGACTTG-3′ | NM_030678.3 |
| | | (SEQ ID NO: 35) | |
| | Reverse | 5′-AGGTGAAGTGGTCTGGAAAGGC-3′ | NM_030678.3 |
| | | (SEQ ID NO: 36) | |
| GAPDH | Forward | 5′-TGGGTGTGAACCATGAGAAG-3′ | XM_029478683.1 |
| | | (SEQ ID NO: 41) | |
| | Reverse | 5′-GCTAAGCAGTTGGTGGTGC-3′ | XM_029478683.1 |
| | | (SEQ ID NO: 42) | |

### 5. Statistical Analysis

All analytical values were represented by mean±SEM. The result was analyzed using the SAS statistical software, version 9.4 or GraphPad Prism 5.0 (GraphPad Software). Student's t-test and one-way analysis variance (ANOVA) were used to compare differences between the test substance-administered groups and the control groups. Furthermore, significance was validated using Duncan's multiple range test after analysis by ANOVA to compare the differences between the control group and the test substance-administered groups. Only p<0.05 was considered statistically significant.

### Test Examples

<Evaluation of Muscle Fatigue-Alleviating Effect in In-Vivo System>—without Exercise

### Test Example 6: Effect on Body Weight of Experimental Animals

The weight of the experimental animals measured once a week during the test period and is shown in Table 16. The experimental animals of all the test groups showed constant increase in body weight during the test period, showing normal body weight changes. There was no significant difference in the body weight between the control group (G1) and all the test substance-administered groups (G2 to G5) during the test period.

TABLE 16

| | | | |
|---|---|---|---|
| Test groups | Week 0 | Week 1 | Week 2 |

| | | | | |
|---|---|---|---|---|
| G1 | — | 31.0 ± 0.5 | 33.5 ± 0.4 | 34.4 ± 0.5 |
| G2 | Example 1 | 31.0 ± 0.3 | 33.2 ± 0.8 | 34.1 ± 0.9 |
| G3 | Example 2 | 31.1 ± 0.1 | 33.5 ± 0.6 | 34.0 ± 0.6 |
| G4 | Example 3 | 31.0 ± 0.4 | 33.2 ± 0.5 | 34.1 ± 0.5 |
| G5 | Example 4 | 30.9 ± 0.2 | 33.3 ± 0.4 | 34.1 ± 0.6 |

### Test Example 7: Effect on Exercise (Forced Swimming) Time

In order to evaluate the effect of alleviating fatigue from exercise of the test substances, a weight corresponding to 5% of the body weight of the experimental animal was attached to the tail and the experimental animal was forced to swim until exhaustion. The result is shown in Table 17.

TABLE 17

| | |
|---|---|
| Test groups | Time to exhaustion (sec) |

| | | |
|---|---|---|
| G1 | — | 1119.2 ± 125.2 |
| G2 | Example 1 | 1193.4 ± 176.8 |
| G3 | Example 2 | 1351.7 ± 201.5** |
| G4 | Example 3 | 1190.3 ± 157.3 |
| G5 | Example 4 | 1189.7 ± 165.4 |

As seen from Table 17, the time to exhaustion increased significantly in the Example 2-administered group (G3) as compared to the control group (G1).

### Test Example 8: Effect on Lactate Concentration after Exercise (Forced Swimming)

The increase in lactate level in the blood during exercise is known to be proportional to the intensity of exercise. Lactate threshold was used as an indicator of aerobic exercise performance. The ability to remove lactate in the blood during exercise or recovery indicates the correlation between exercise performance and capacity.

The result of investigating the effect of the administration of the test substances on blood lactate levels after exercise (forced swimming) is shown in Table 18.

TABLE 18

| | | Lactate concentration (μmol/L) before and after exercise | | | |
|---|---|---|---|---|---|
| | | (forced swimming) | | | |
| | | Immediately before | Immediately after forced swimming | | |
| Test groups | | forced swimming | 0 min | 10 min | 30 min |
| G1 | — | 1.29 ± 0.06 | 14.78 ± 1.22 | 11.13 ± 0.77 | 8.32 ± 1.40 |
| G2 | Example 1 | 2.27 ± 0.07 | 12.94 ± 0.56 | 10.09 ± 1.28 | 8.68 ± 1.54 |
| G3 | Example 2 | 2.11 ± 0.22 | 12.11 ± 0.18 | 8.64 ± 1.12* | 6.12 ± 1.26* |
| G4 | Example 3 | 2.21 ± 0.06 | 12.77 ± 0.24 | 10.14 ± 1.17 | 8.89 ± 1.51 |
| G5 | Example 4 | 2.20 ± 0.05 | 12.86 ± 0.37 | 10.16 ± 1.31 | 8.88 ± 1.48 |

As seen from Table 18, the blood lactate levels of all the test groups peaked immediately after the exercise (when exhausted) as compared to before the exercise, and the blood lactate levels decreased over time. In particular, the composition of Example 2 significantly decreased the blood lactate level after the exercise.

### Test Example 9: Effect on Serum Markers

The result of measuring the contents of lactate, BUN and CREA and the activities of CK, LDH, ALT and AST in the serum taken after forced swimming for 60 minutes without weight load is shown in Table 19.

TABLE 19

| | G1 | G2 | G3 | G4 | G5 |
|---|---|---|---|---|---|
| | — | Example 1 | Example 2 | Example 3 | Example 4 |
| Lactate (μmol/L) | 5.35 ± 0.33 | 4.53 ± 0.49* | 3.42 ± 0.36** | 4.52 ± 0.27* | 4.46 ± 0.52* |
| LDH (U/L) | 1030.7 ± 124.6 | 1224.3 ± 162.1 | 912.6 ± 104.5* | 1216.4 ± 117.5 | 1221.5 ± 121.7 |
| BUN (mg/dL) | 9.54 ± 0.72 | 8.15 ± 0.68 | 8.26 ± 0.64 | 8.07 ± 0.14 | 8.11 ± 0.19 |
| CREA (mg/dL) | 0.34 ± 0.01 | 0.35 ± 0.01 | 0.33 ± 0.01 | 0.34 ± 0.02 | 0.35 ± 0.01 |
| CK (U/L) | 1094.5 ± 260.5 | 1896.1 ± 196.4 | 1124.7 ± 181.4 | 1765.8 ± 118.2 | 1871.4 ± 213.7 |
| ALT (U/L) | 57.88 ± 12.10 | 100.41 ± 19.75 | 67.86 ± 15.45 | 95.13 ± 14.59 | 97.24 ± 13.68 |
| AST (U/L) | 106.45 ± 15.81 | 160.81 ± 20.53 | 124.15 ± 14.19 | 157.46 ± 14.87 | 162.31 ± 15.88 |

Lactate is produced in tissue as pyruvate is reduced during anaerobic glycolysis. It is used as an indicator of fatigue caused by exercise many studies since it reduces exercise performance by acidifying the environment in muscle cells and inhibiting the activity of phosphorylase and myosin ATPase. Lactate dehydrogenase (LDH) is an enzyme that catalyzes the formation of lactate from pyruvate. An excessive amount of pyruvate is produced during high-intensity exercise and, as a result, the activity of LDH that catalyzes the process of converting it into lactate is increased. Therefore, the increased activity of LDH in serum is an indicator of muscle damage due to increased stress on skeletal muscle.

As seen from Table 19, the lactate concentration in the serum decreased significantly as compared to the control group (G1) in the groups to which the compositions of the examples were administered (G2 to G4), and the activity of LDH in the serum decreased significantly in the group to which the composition of the example was administered (G2) as compared to the control group (G1).

<Evaluation of Exercise Performance-Improving Effect in In-Vivo System>—with Exercise

### Test Example 10: Body Weight of Experimental Animals

The weight of the experimental animals was measured once in 2 weeks during the test period. The result is shown in Table 20.

TABLE 20

| | | Body weight of experimental animals (g) | | | |
|---|---|---|---|---|---|
| Test groups | | Week 0 | Week 2 | Week 4 | Week 6 |
| G1 | — | 26.9 ± 0.3 | 34.8 ± 0.5 | 39.0 ± 0.8 | 42.5 ± 0.7 |
| G2 | Example 1 | 26.6 ± 0.4 | 32.7 ± 0.7 | 35.5 ± 1.0 | 37.9 ± 1.2 |
| G3 | Example 2 | 26.6 ± 0.1 | 32.5 ± 0.3 | 35.0 ± 0.7 | 37.4 ± 0.8 |
| G6 | — | 26.6 ± 0.2 | 31.8 ± 0.4 | 35.4 ± 0.5 | 38.7 ± 0.5 |
| G7 | Example 1 | 26.5 ± 0.2 | 30.5 ± 0.4 | 33.0 ± 0.5 | 36.6 ± 0.7 |
| G8 | Example 2 | 26.6 ± 0.3 | 30.4 ± 0.3 | 33.0 ± 0.5 | 36.1 ± 0.8 |
| G9 | Example 3 | 26.5 ± 0.2 | 30.3 ± 0.4 | 33.1 ± 0.4 | 36.5 ± 0.5 |
| G10 | Example 4 | 26.4 ± 0.2 | 30.4 ± 0.2 | 32.9 ± 0.4 | 36.4 ± 0.4 |
| G11 | Cr | 26.8 ± 0.3 | 31.2 ± 0.6 | 34.0 ± 0.8 | 38.0 ± 1.0 |

As seen from Table 20, the experimental animals of all the groups showed constant increase in body weight during the test period, showing normal body weight changes. As compared to the non-exercise control group (G1), the body weight of the group to which the composition of the example was administered (G2) was decreased significantly from week 4 to the end of the test, and the body weight of the exercise control group (G6) was decreased significantly from week 1 to the end of the test as compared to the non-exercise control group (G1). Among the exercise test groups, the groups to which the compositions of the examples were administered (G7 to G10) showed significant decrease in body weight from week 2 as compared to the exercise control group (G6).

### Test Example 11: Effect on Exercise Time and Exercise Capacity Until Exhaustion

In order to evaluate the performance of the effect of the test substances on endurance exercise performance, exercise was started with 10 degrees of slope at 10 m/min for 5 minutes, and the intensity of the exercise was increased by 1 m/min every 1 minute up to the highest speed of 25 m/min. The result is shown in Table 21.

TABLE 21

| | | Exercise time and capacity until exhaustion (g) | |
|---|---|---|---|
| Test groups | | Time until exhaustion (sec) | Exercise capacity |
| G1 | — | 1,119 ± 58 | 1,217 ± 127 |
| G2 | Example 1 | 1,472 ± 66* | 1,583 ± 91* |
| G3 | Example 2 | 1,675 ± 49** | 1,794 ± 87** |
| G4 | Example 3 | 1,463 ± 75* | 1,559 ± 43* |
| G5 | Example 4 | 1,531 ± 64* | 1,611 ± 84* |
| G6 | — | 1,950 ± 85*** | 2,406 ± 123*** |
| G7 | Example 1 | 2,393 ± 93⁺ | 2,832 ± 119⁺ |
| G8 | Example 2 | 2,850 ± 74⁺⁺⁺ | 3,710 ± 51⁺⁺⁺ |
| G9 | Example 3 | 2,384 ± 67⁺ | 2,716 ± 75⁺ |
| G10 | Example 4 | 2,274 ± 91⁺ | 2,893 ± 48⁺ |
| G11 | Cr | 2,674 ± 177⁺⁺ | 3,668 ± 279⁺⁺ |

*, ** and ***indicate significant differences with p < 0.05, p < 0.01 and p < 0.001, respectively, as compared to the G1 group (G2 to G5, G6).
⁺, ⁺⁺ and ⁺⁺⁺indicate significant differences with p < 0.05, p < 0.01 and p < 0.001, respectively, as compared to the G6 group (G7 to G11).

As shown in Table 21, the exercise time until exhaustion was the shortest for the non-exercise control group (G1) as 1119±58 seconds as compared to other test groups. The exercise time until exhaustion increased significantly in the non-exercise test groups to which the compositions of the examples were administered (G2 to G5) and the exercise control group (G6) as compared to the non-exercise control group (G1). For the exercise test groups, the groups to which the compositions of the examples were administered 10 (G7 to G10) and the Cr-administered group (G11) showed significantly increased exercise time until exhaustion as compared to the exercise control group (G6).

As a result of the exercise capacity of the experimental animals, the exercise capacity of the non-exercise groups to which the compositions of the examples were administered (G2 to G5) was increased significantly as compared to the non-exercise control group (G1). The exercise capacity of the exercise control group (G6) increased significantly as compared to the non-exercise control group (G1) and, for the exercise test groups, the exercise capacity of the groups to which the compositions of the examples were administered (G7 to G10) and the Cr control group (G11) increased significantly as compared to the exercise control group (G6).

This indicates that the administration of the compositions of the examples has an effect of improving exercise performance.

### Test Example 12: Weight of Liver and Muscles

After the end of the test, the weight of the muscles and liver of the experimental animals was measured. The result is shown in Table 22.

TABLE 22

| | | Weight of liver and muscles (g) | | | | |
|---|---|---|---|---|---|---|
| | | | | | Extensor | |
| | | Quadriceps | | Soleus | digitorum | |
| | | femoris | Gastrocnemius | muscle | longus muscle | |
| Test groups | | muscle (QF) | muscle (GA) | (SOL) | (EDL) | Liver |
| G1 | — | 0.283 ± | 0.400 ± 0.013 | 0.021 ± | 0.020 ± 0.001 | 2.16 ± |
| | | 0.017 | | 0.001 | | 0.07 |
| G2 | Example 1 | 0.282 ± | 0.392 ± 0.010 | 0.021 ± | 0.021 ± 0.001 | 1.87 ± |
| | | 0.014 | | 0.001 | | 0.08 |
| G3 | Example 2 | 0.284 ± | 0.396 ± 0.015 | 0.022 ± | 0.022 ± 0.001 | 1.88 ± |
| | | 0.013 | | 0.001 | | 0.06 |
| G6 | — | 0.289 ± | 0.404 ± 0.007 | 0.024 ± | 0.024 ± 0.001 | 1.98 ± |
| | | 0.015 | | 0.001 | | 0.05 |
| G7 | Example 1 | 0.296 ± | 0.406 ± 0.012 | 0.023 ± | 0.020 ± 0.001 | 1.83 ± |
| | | 0.025 | | 0.001 | | 0.07 |
| G8 | Example 2 | 0.298 ± | 0.410 ± 0.011 | 0.024 ± | 0.022 ± 0.001 | 1.85 ± |
| | | 0.021 | | 0.001 | | 0.06 |
| G9 | Example 3 | 0.295 ± | 0.405 ± 0.013 | 0.023 ± | 0.021 ± 0.001 | 1.84 ± |
| | | 0.024 | | 0.001 | | 0.08 |
| G10 | Example 4 | 0.294 ± | 0.406 ± 0.008 | 0.024 ± | 0.021 ± 0.001 | 1.85 ± |
| | | 0.023 | | 0.001 | | 0.07 |
| G11 | Cr | 0.330 ± | 0.409 ± 0.012 | 0.023 ± | 0.022 ± 0.001 | 2.08 ± |
| | | 0.026 | | 0.001 | | 0.05 |

Since the body weight of the experimental animals was decreased due to exercise and the administration of the test substances, the relative weight of the muscles and liver per 100 g of body weight was calculated and shown in Table 23.

TABLE 23

| | | Relative weight of liver and muscles (g/100 g body weight) | | | | |
|---|---|---|---|---|---|---|
| | | | | | Extensor | |
| | | Quadriceps | | Soleus | digitorum | |
| | | femoris | Gastrocnemius | muscle | longus muscle | |
| Test groups | | muscle (QF) | muscle (GA) | (SOL) | (EDL) | Liver |
| G1 | — | 0.668 ± | 0.940 ± 0.031 | 0.049 ± | 0.047 ± 0.001 | 5.08 ± |
| | | 0.040 | | 0.001 | | 0.15 |
| G2 | Example 1 | 0.742 ± | 1.040 ± 0.036 | 0.056 ± | 0.056 ± 0.002* | 4.92 ± |
| | | 0.031 | | 0.002* | | 0.10 |
| G3 | Example 2 | 0.747 ± | 1.094 ± 0.024* | 0.060 ± | 0.059 ± 0.001* | 4.95 ± |
| | | 0.033* | | 0.001* | | 0.11 |
| G6 | — | 0.744 ± | 1.044 ± 0.019* | 0.062 ± | 0.063 ± 0.001* | 5.12 ± |
| | | 0.036 | | 0.002* | | 0.18 |
| G7 | Example 1 | 0.827 ± | 1.122 ± 0.032⁺ | 0.064 ± | 0.056 ± 0.003 | 5.07 ± |
| | | 0.080⁺ | | 0.001 | | 0.19 |
| G8 | Example 2 | 0.832 ± | 1.211 ± 0.024⁺⁺ | 0.068 ± | 0.064 ± 0.001 | 5.10 ± |
| | | 0.064⁺ | | 0.002 | | 0.09 |
| G9 | Example 3 | 0.823 ± | 1.120 ± 0.016⁺ | 0.065 ± | 0.058 ± 0.002 | 5.11 ± |
| | | 0.075⁺ | | 0.001 | | 0.17 |
| G10 | Example 4 | 0.825 ± | 1.124 ± 0.021⁺ | 0.064 ± | 0.055 ± 0.002 | 5.12 ± |
| | | 0.057⁺ | | 0.002 | | 0.19 |
| G11 | Cr | 0.873 ± | 1.078 ± 0.028 | 0.061 ± | 0.059 ± 0.004 | 5.49 ± |
| | | 0.067⁺ | | 0.002 | | 0.13 |

As seen from Table 23, the relative weight of the gastrocnemius muscle (GA), the soleus muscle (SOL) and the extensor digitorum longus muscle (EDL) increased significantly in the exercise control group (G6) as compared to the non-exercise control group (G1). Furthermore, the relative weight of the soleus muscle (SOL) and the extensor digitorum longus muscle (EDL) increased significantly in the group to which the composition of the example was administered (G2) compared to the non-exercise control group (G1). In addition, for the exercise test groups, the relative weight of the quadriceps femoris muscle (QF) and the gastrocnemius muscle (GA) increased significantly in the groups to which the compositions of the examples were administered (G7 to G10) as compared to the exercise control group (G6).

That is to say, it can be seen that the administration of the compositions of the present disclosure provides muscle-augmenting effect.

### Test Example 13: Analysis of Blood Biochemistry

The result of measuring the contents of lactate, CREA and BUN and the activities of CK, LDH, ALT, AST and ALP in the serum collected at the end of the test is shown in Table 24.

TABLE 24

| | | Serum analysis | | | | |
|---|---|---|---|---|---|---|
| | | | Lactate | | BUN | CREA |
| Test groups | | CK (U/L) | (μmol/L) | LDH (U/L) | (mg/dL) | (mg/dL) |
| G1 | — | 393.2 ± | 6.51 ± 0.47 | 1,009 ± | 8.57 ± 0.41 | 0.335 ± |
| | | 80.6 | | 77 | | 0.014 |
| G2 | Example 1 | 416.7 ± | 5.15 ± 0.30* | 894 ± | 7.54 ± 0.47 | 0.312 ± |
| | | 65.1 | | 94* | | 0.008 |
| G3 | Example 2 | 384.5 ± | 4.80 ± 0.53** | 815 ± | 7.23 ± 0.31 | 0.308 ± |
| | | 45.3 | | 45* | | 0.005 |
| G6 | — | 552.8 ± | 7.68 ± 0.61 | 1,569 ± | 8.39 ± 0.34 | 0.324 ± |
| | | 64.0 | | 91 | | 0.015 |
| G7 | Example 1 | 471.7 ± | 6.27 ± 0.31⁺ | 1,198 ± | 7.10 ± 0.40 | 0.348 ± |
| | | 63.2⁺ | | 51⁺ | | 0.006 |
| G8 | Example 2 | 324.6 ± | 5.76 ± 0.23⁺⁺ | 1,007 ± | 6.89 ± 0.31⁺⁺ | 0.342 ± |
| | | 27.4⁺⁺⁺ | | 49⁺⁺ | | 0.011 |
| G9 | Example 3 | 468.4 ± | 6.20 ± 0.36⁺ | 1,186 ± | 7.26 ± 0.29 | 0.338 ± |
| | | 45.7⁺ | | 56⁺ | | 0.005 |
| G10 | Example 4 | 476.3 ± | 6.19 ± 0.42⁺ | 1,245 ± | 7.15 ± 0.38 | 0.346 ± |
| | | 31.9⁺ | | 53⁺ | | 0.012 |
| G11 | Cr | 521.5 ± | 5.30 ± 0.41⁺⁺ | 1,169 ± | 7.84 ± 0.47 | 0.363 ± |
| | | 68.3 | | 103⁺ | | 0.006 |

As seen from Table 24, the activities of CK and LDH and the concentrations of lactate and BUN (blood urea nitrogen) in the serum were decreased significantly in the groups to which the compositions of the examples were administered (G7 to G10) as compared to the exercise control group (G6).

Thus, it can be seen from the above results that the administration of the compositions of the examples of the present disclosure helps to alleviate muscle fatigue.

### Test Example 14: Glycogen Content in Liver and Muscle (Gastrocnemius Muscle, GA)

Carbohydrates, which are one of the most important energy sources during exercise, provide energy for muscle contraction via the glycogen breakdown process. If blood glucose level decreases during exercise, glycogen stored in the liver or muscle is used directly as an energy source. Therefore, the inhibiting of glycogen breakdown, i.e. the saving of glycogen, means that muscle contraction can be maintained for a long time.

Accordingly, the glycogen content in the muscle (GA) collected at the end of the test was measured. The result is shown in Table 25.

TABLE 25

| | | Glycogen content in 1 mg | Total glycogen content |
|---|---|---|---|
| Test groups | | of GA (μg/mg GA) | in GA (μg/GA) |
| G1 | — | 3.20 ± 0.22 | 1285.5 ± 113.1 |
| G2 | Example 1 | 3.56 ± 0.15* | 1442.1 ± 76.2* |
| G6 | — | 2.83 ± 0.14 | 1145.8 ± 62.4 |
| G7 | Example 1 | 3.38 ± 0.12⁺ | 1404.4 ± 83.3⁺ |
| G8 | Example 2 | 4.23 ± 0.16⁺⁺ | 2031.1 ± 72.6⁺⁺ |
| G9 | Example 3 | 3.29 ± 0.11⁺ | 1341.5 ± 64.1⁺ |
| G10 | Example 4 | 3.31 ± 0.13⁺ | 1391.3 ± 62.8⁺ |
| G11 | Cr | 3.32 ± 0.21⁺ | 1350.5 ± 73.7⁺ |

As seen from Table 25, the glycogen content in the muscle (GA) tissue increased significantly in the exercise test groups to which the compositions of the examples were administered (G7 to G10) as compared to the exercise control group (G6).

From the above results, it can be seen that the compositions of the examples of the present disclosure can be energy sources that help to alleviate muscle fatigue and improve muscular endurance since they increase the glycogen content in the muscle (GA) tissue.

### Test Example 15: Change in Expression of Proteins in Muscle (Gastrocnemius Muscle, GA)

In this study, western blot was performed using a muscle (gastrocnemius muscle, GA) tissue lysate in order to investigate the effect of the administration of the test substances on change in the expression of proteins associated with PGC-1α activation. Then, the activation of the proteins was evaluated based on the western blot analysis result. The result is shown in Table 26.

TABLE 26

| | | p-PGC1α/PGC1α | p-AMPK/AMPK | p-p38/p38 | p-Nrf2/Nrf2 |
|---|---|---|---|---|---|
| Test groups | | ratio | ratio | ratio | ratio |
| G1 | — | 1.00 ± 0.00 | 1.00 ± 0.00 | 1.00 ± 0.00 | 1.00 ± 0.00 |
| G2 | Example 1 | 1.14 ± 0.07* | 1.22 ± 0.09* | 1.71 ± 0.16* | 1.02 ± 0.06 |
| G6 | — | 1.38 ± 0.42** | 1.33 ± 0.09** | 0.84 ± 0.08 | 1.52 ± 0.05** |
| G7 | Example 1 | 1.98 ± 0.34⁺ | 1.41 ± 0.14 | 1.86 ± 0.13⁺ | 1.30 ± 0.16 |
| G8 | Example 2 | 2.44 ± 0.23⁺⁺ | 1.83 ± 0.27⁺ | 2.24 ± 0.31⁺⁺ | 1.64 ± 0.22 |
| G9 | Example 3 | 1.86 ± 0.19⁺ | 1.40 ± 0.08 | 1.75 ± 0.11⁺ | 1.28 ± 0.08 |
| G10 | Example 4 | 1.80 ± 0.24⁺ | 1.38 ± 0.20 | 1.80 ± 0.17⁺ | 1.31 ± 0.06 |
| G11 | Cr | 2.28 ± 0.84⁺⁺ | 1.43 ± 0.13 | 1.94 ± 0.16⁺ | 1.35 ± 0.17 |

As shown in Table 26, the activity of proteins associated with the activation of PGC-1α increased in the groups to which the compositions of the examples were administered. In particular, for the exercise test groups, the groups to which the compositions of the examples were administered (G7 to G10) showed significantly increased activity of PGC1α, AMPK and p-38 MAPK as compared to the exercise control group (G6).

Through these results, it can be inferred that the administration of the compositions of the examples together with regular exercise training can significantly increase the activation of AMPK and p38 MAPK, which are proteins associated with the activation of PGC-1α, thereby regulating the expression of the genes involved in mitochondrial biosynthesis and sugar metabolism and improving exercise performance.

### Test Example 16: Change in mRNA Expression in Muscle (Soleus Muscle, SOL)

Muscle can be classified into slow muscle and fast muscle depending on the physiological rate of contraction. Since the slow muscle has a large number of mitochondria and is highly active and resistant to fatigue, it enables exercise for long time. Therefore, the effect of endurance exercise training and the administration of the test substances on the expression of antioxidant-related genes (SOD2, GPx1, UCP2, UCP3), LDH-related genes (ERRα, LDH B, MCT1), mitochondrial synthesis-related genes (Tfam, CPT-1β, mtDNA, NRF1) and energy metabolism-related genes (PGC-1α, GYS, PPARγ, PPARδ) in the soleus muscle, which is a slow muscle, was investigated. The result is shown in Tables 27 to 30.

TABLE 27
Change in mRNA expression of antioxidant-related genes

| Test groups | | SOD2 | GPx1 | UCP2 | UCP3 |
|---|---|---|---|---|---|
| G1 | — | 1.00 ± 0.00 | 1.00 ± 0.00 | 1.00 ± 0.00 | 1.00 ± 0.00 |
| G2 | Example 1 | 1.16 ± 0.03* | 0.89 ± 0.10* | 1.23 ± 0.05* | 0.51 ± 0.09 |
| G6 | — | 1.11 ± 0.03* | 1.15 ± 0.13 | 1.50 ± 0.46* | 0.69 ± 0.22 |
| G7 | Example 1 | 1.47 ± 0.13⁺ | 3.28 ± 0.36⁺⁺ | 6.91 ± 1.47⁺⁺ | 0.64 ± 0.10 |
| G8 | Example 2 | 1.73 ± 0.22⁺⁺ | 4.57 ± 0.29⁺⁺⁺ | 7.64 ± 1.50⁺⁺ | 0.73 ± 0.11 |
| G9 | Example 3 | 1.45 ± 0.11⁺ | 3.05 ± 0.16⁺⁺ | 6.84 ± 1.06⁺⁺ | 0.60 ± 0.13 |
| G10 | Example 4 | 1.40 ± 0.15⁺ | 3.19 ± 0.28⁺⁺ | 6.85 ± 1.13⁺⁺ | 0.62 ± 0.12 |
| G11 | Cr | 1.52 ± 0.08⁺ | 1.92 ± 0.29⁺ | 14.96 ± 1.96⁺⁺⁺ | 0.87 ± 0.22 |

From Table 27, it can be seen that the activity of the proteins associated with antioxidation was increased in the groups to which the compositions of the examples were administered. In particular, for the exercise test groups, the activity of SOD2, GPx1 and UCP2 was significantly increased in the groups to which the compositions of the examples were administered (G7 to G10) as compared to the exercise control group (G6).

Through these results, it can be inferred that the administration of the compositions of the examples along with regular exercise training can inhibit damage to muscle cells by protecting mitochondria from oxidative stress by increasing the mRNA expression of SOD2, GPx1 and UCP2.

TABLE 28
Change in mRNA expression of LDH-related genes

| Test groups | | ERRα | LDH B | MCT1 |
|---|---|---|---|---|
| G1 | — | 1.00 ± 0.00 | 1.00 ± 0.00 | 1.00 ± 0.00 |
| G2 | Example 1 | 1.67 ± 0.22** | 1.76 ± 0.49** | 0.41 ± 0.03** |
| G6 | — | 1.24 ± 0.32* | 1.20 ± 0.22* | 0.28 ± 0.07** |
| G7 | Example 1 | 1.41 ± 0.22 | 1.26 ± 0.46 | 0.56 ± 0.25⁺ |
| G8 | Example 2 | 1.52 ± 0.16⁺ | 1.35 ± 0.31⁺ | 0.64 ± 0.13⁺ |
| G9 | Example 3 | 1.40 ± 0.21 | 1.25 ± 0.24 | 0.50 ± 0.12⁺ |
| G10 | Example 4 | 1.44 ± 0.11 | 1.27 ± 0.13 | 0.57 ± 0.20⁺ |
| G11 | Cr | 0.58 ± 0.19⁺ | 0.35 ± 0.11⁺ | 0.99 ± 0.31⁺⁺ |

As seen from Table 28, among the LDH-related genes, the mRNA expression of ERRα and LDH B was increased and the mRNA expression of MCT1 was increased significantly in the groups to which the compositions of the examples were administered.

TABLE 29
Change in mRNA expression of mitochondrial synthesis-related genes

| Test groups | | mtDNA | Tfam | CPT-1β | NRF1 |
|---|---|---|---|---|---|
| G1 | — | 1.00 ± 0.00 | 1.00 ± 0.00 | 1.00 ± 0.00 | 1.00 ± 0.00 |
| G2 | Example 1 | 1.29 ± 0.12* | 4.74 ± 0.32*** | 0.34 ± 0.02* | 15.52 ± 2.59*** |
| G6 | — | 1.52 ± 0.09** | 1.47 ± 0.14* | 0.38 ± 0.03*** | 4.92 ± 0.95** |
| G7 | Example 1 | 4.66 ± 0.64⁺⁺ | 1.98 ± 0.42⁺ | 0.86 ± 0.04⁺⁺ | 29.20 ± 3.10⁺⁺ |
| G8 | Example 2 | 5.78 ± 0.57⁺⁺⁺ | 2.17 ± 0.34⁺⁺ | 0.89 ± 0.02⁺⁺ | 31.34 ± 3.06⁺⁺ |
| G9 | Example 3 | 4.57 ± 0.45⁺⁺ | 1.81 ± 0.27⁺ | 0.81 ± 0.03⁺⁺ | 28.31 ± 0.82⁺⁺ |
| G10 | Example 4 | 4.46 ± 0.29⁺⁺ | 1.94 ± 0.14⁺ | 0.79 ± 0.06⁺⁺ | 28.19 ± 0.57⁺⁺ |
| G11 | Cr | 1.69 ± 0.20 | 1.34 ± 0.07 | 0.95 ± 0.28⁺⁺ | 9.17 ± 2.18 |

From Table 29, it can be seen that the expression of the genes associated with mitochondrial synthesis tend to increase in the groups to which the compositions of the examples were administered. In particular, for the exercise test groups, the mRNA expression of mtDNA, Tfam CPT-1β and NRF1 increased significantly in the groups to which the compositions of the examples were administered (G7 to G10) as compared to the exercise control group (G6).

Through these results, it can be inferred that the administration of the compositions of the examples along with regular exercise training can enhance exercise performance by increasing the mRNA expression of mtDNA, Tfam CPT-1β and NRF1 and, thereby, increasing mitochondrial biosynthesis.

TABLE 30
Change in mRNA expression of energy metabolism-related genes

| Test groups | | PGC1α | PPARγ | PPARδ | GSY |
|---|---|---|---|---|---|
| G1 | — | 1.00 ± 0.00 | 1.00 ± 0.00 | 1.00 ± 0.00 | 1.00 ± 0.00 |
| G2 | Example 1 | 0.88 ± 0.08 | 0.25 ± 0.00 | 2.87 ± 0.95 | 2.60 ± 0.51 |
| G6 | — | 2.34 ± 0.32** | 0.54 ± 0.12* | 1.44 ± 0.28 | 0.40 ± 0.11 |
| G7 | Example 1 | 2.73 ± 0.51 | 5.82 ± 1.27⁺ | 2.42 ± 0.45⁺ | 0.27 ± 0.06 |
| G8 | Example 2 | 2.89 ± 0.42 | 6.98 ± 1.16⁺⁺ | 3.81 ± 0.27⁺⁺ | 0.31 ± 0.03 |
| G9 | Example 3 | 2.62 ± 0.40 | 5.64 ± 1.20⁺ | 2.24 ± 0.16⁺ | 0.21 ± 0.05 |
| G10 | Example 4 | 2.68 ± 0.37 | 5.57 ± 1.08⁺ | 2.37 ± 0.21⁺ | 0.22 ± 0.03 |
| G11 | Cr | 5.09 ± 1.22⁺ | 0.29 ± 0.05 | 3.82 ± 0.90⁺⁺ | 0.92 ± 0.31 |

From Table 30, it can be seen that the expression of the energy metabolism-related genes tend to increase in the groups to which the compositions of the examples were administered. In particular, for the exercise test groups, the groups to which the compositions of the examples were administered (G7 to G10) showed significant increase in the expression of PPARγ and PPARd as compared to the exercise control group (G6).

Through these results, it can be inferred that the administration of the compositions of the examples along with regular exercise training can enhance exercise performance by increasing the mRNA expression of PGC1α, PPARγ and PPARδ and, thereby, increasing energy metabolism.

In conclusion, it is though that the administration of the compositions of the examples combined with endurance exercise training will exhibit muscle-augmenting effect, help to improve muscle fatigue by decreasing lactate level increased during exercise, and improve exercise performance by regulating the expression of the genes involved in mitochondrial biosynthesis and sugar metabolism. Therefore, the compositions of the examples can be used as functional substances that improve exercise performance during endurance exercise.

### Preparation Examples

### Preparation Example 1: Preparation of Tablet

10 mg of any composition selected from Example 1 to Example 4 was mixed with 9 mg of vitamin E, 9 mg of vitamin E, 9 mg of vitamin C, 200 mg of galactooligosaccharides, 60 mg of lactose and 140 mg of maltose and granulated using a fluidized-bed dryer. Then, 6 mg of sugar ester was added. 500 mg of the resulting composition was prepared into a tablet by a common method.

### Preparation Example 2: Preparation of Capsule

According to a common soft capsule preparation method, 10 mg of any composition selected from Example 1 to Example 4 was mixed with 9 mg of vitamin C, 2 mg of palm oil, 8 mg of hydrogenated vegetable oil, 4 mg of yellow beeswax and 9 mg of lecithin. The mixture was filled in a gelatin capsule to produce a soft capsule.

### Preparation Example 3: Preparation of Pill

5 mg of any composition selected from Example 1 to Example 4 was mixed appropriately with honey, dextrin, starch, microcrystalline cellulose, calcium CMC, etc. and then prepared into a pill.

### Preparation Example 4: Preparation of Drink

20 mg of any composition selected from Example 1 to Example 4 was mixed with 9 mg of vitamin E, 9 mg of vitamin C, 10 g of glucose, 0.6 g of citric acid and 25 g of oligosaccharide syrup. Then, after adding 300 mL of purified water, 200 ml of the mixture was filled per bottle. Then, a drink was prepared by sterilizing the bottle at 130° C. for 4 to 5 seconds.

### Preparation Example 5: Preparation of Granule

5 mg of any composition selected from Example 1 to Example 4 was mixed with 9 mg of vitamin E, 9 mg of vitamin C, 250 mg of anhydrous crystalline glucose and 550 mg of starch. After forming the mixture into a granule using a fluidized-bed granulator, a granule was prepared by filling in a pouch.

From the above description, those having ordinary skill in the art to which the present disclosure belongs will be able to understand that the present disclosure can be embodied in other specific forms without changing its technical idea or essential features. In this regard, it is to be understood that the above-described exemplary embodiments are only exemplary and all not limitative in any way. It is to be understood that the scope of the present disclosure includes all changes or modifications derived from the attached claims and their equivalents.

1. A health functional food composition for improving exercise performance, comprising a gypenoside compound represented by Chemical Formula 1, a stereoisomer thereof or a sitologically acceptable salt thereof as an active ingredient:

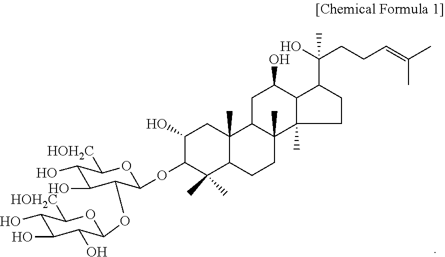

2. The health functional food composition according to claim 1, wherein the active ingredient is gypenoside L or gypenoside LI.

3. The health functional food composition according to claim 1, wherein the active ingredient is gypenoside L and gypenoside LI.

4. The health functional food composition according to claim 3, wherein the weight ratio of gypenoside L and gypenoside LI is 100:20 to 80.

5. The health functional food composition according to claim 1, wherein the administration dosage of the active ingredients is 0.01 to 200 mg/kg/day.

6. A pharmaceutical composition for improving exercise performance, comprising a gypenoside compound represented by Chemical Formula 1, a stereoisomer thereof or a pharmaceutically acceptable salt thereof as an active ingredient: [chemical structure]


SEQUENCE LISTING (50 sequences)

SEQ ID NO: 1 - RNA, 20 nt, synthetic construct
  source 1..20: mol_type = other RNA
atcaggacccattgcaagga

SEQ ID NO: 2 - RNA, 21 nt, synthetic construct
  source 1..21: mol_type = other RNA
aggtttcacttcttgcaagct

SEQ ID NO: 3 - RNA, 19 nt, synthetic construct
  source 1..19: mol_type = other RNA
caggtcggacgtacttgag

SEQ ID NO: 4 - RNA, 19 nt, synthetic construct
  source 1..19: mol_type = other RNA
caggtcggacgtacttgag

SEQ ID NO: 5 - RNA, 21 nt, synthetic construct
  source 1..21: mol_type = other RNA
ctcgtcttgccgattgaaggt

SEQ ID NO: 6 - RNA, 21 nt, synthetic construct
  source 1..21: mol_type = other RNA
tctgcaatgcaggcagctgtc

SEQ ID NO: 7 - RNA, 20 nt, synthetic construct
  source 1..20: mol_type = other RNA
gcctacagaaccatcgccag

SEQ ID NO: 8 - RNA, 20 nt, synthetic construct
  source 1..20: mol_type = other RNA
gccaccatcttcagcataca

SEQ ID NO: 9 - RNA, 18 nt, synthetic construct
  source 1..18: mol_type = other RNA
ttcggcgactgcaagctc

SEQ ID NO: 10 - RNA, 22 nt, synthetic construct
  source 1..22: mol_type = other RNA
cacagcctcagcatcttcaatg

SEQ ID NO: 11 - RNA, 22 nt, synthetic construct
  source 1..22: mol_type = other RNA
cctcagatcgtcaagtacagcc

SEQ ID NO: 12 - RNA, 22 nt, synthetic construct
  source 1..22: mol_type = other RNA
atccgcttccaatcacacggtg

SEQ ID NO: 13 - RNA, 20 nt, synthetic construct
  source 1..20: mol_type = other RNA
gctgggcagtggtaattgga

SEQ ID NO: 14 - RNA, 24 nt, synthetic construct
  source 1..24: mol_type = other RNA
cagtaattgatttgggaaatgcat

SEQ ID NO: 15 - RNA, 20 nt, synthetic construct
  source 1..20: mol_type = other RNA
ataggcaccgtattgcgtga

SEQ ID NO: 16 - RNA, 20 nt, synthetic construct
  source 1..20: mol_type = other RNA
ctgatagacgaggggatgcg

SEQ ID NO: 17 - RNA, 20 nt, synthetic construct
  source 1..20: mol_type = other RNA
cctggaagaaacgcctgatt

SEQ ID NO: 18 - RNA, 20 nt, synthetic construct
  source 1..20: mol_type = other RNA
cagggtttggcgaaagaaga

SEQ ID NO: 19 - RNA, 21 nt, synthetic construct
  source 1..21: mol_type = other RNA
cacgatcaactgaagcagcaa

SEQ ID NO: 20 - RNA, 21 nt, synthetic construct
  source 1..21: mol_type = other RNA
acgatggccaggaggataatt

SEQ ID NO: 21 - RNA, 20 nt, synthetic construct
  source 1..20: mol_type = other RNA
cgctccacgcaccctcactt

SEQ ID NO: 22 - RNA, 19 nt, synthetic construct
  source 1..19: mol_type = other RNA
gtccatcacccctggagac

SEQ ID NO: 23 - RNA, 22 nt, synthetic construct
  source 1..22: mol_type = other RNA
gctggaagatgagtgctcagag

SEQ ID NO: 24 - RNA, 22 nt, synthetic construct
  source 1..22: mol_type = other RNA
tccaaaccagccatctcctctg

SEQ ID NO: 25 - RNA, 23 nt, synthetic construct
  source 1..23: mol_type = other RNA
ccattcagagcaaagatgcaggg

SEQ ID NO: 26 - RNA, 21 nt, synthetic construct
  source 1..21: mol_type = other RNA
gcataacgctctttgaggttg

SEQ ID NO: 27 - RNA, 21 nt, synthetic construct
  source 1..21: mol_type = other RNA
gctagggtgagggagcttgaa

SEQ ID NO: 28 - RNA, 19 nt, synthetic construct
  source 1..19: mol_type = other RNA
agacccttgacggcttcga

SEQ ID NO: 29 - RNA, 20 nt, synthetic construct
  source 1..20: mol_type = other RNA
gtccttcctccatgcctgac

SEQ ID NO: 30 - RNA, 20 nt, synthetic construct
  source 1..20: mol_type = other RNA
gactgcggttgtgtatggga

SEQ ID NO: 31 - RNA, 22 nt, synthetic construct
  source 1..22: mol_type = other RNA
ggactacttgcactccgagaag

SEQ ID NO: 32 - RNA, 22 nt, synthetic construct
  source 1..22: mol_type = other RNA
catagtggcaccgtccttgatc

SEQ ID NO: 33 - RNA, 25 nt, synthetic construct
  source 1..25: mol_type = other RNA
atgaggtgaccatgaaggagatggc

SEQ ID NO: 34 - RNA, 24 nt, synthetic construct
  source 1..24: mol_type = other RNA
ctggtttctgatgcgctcttggtt

SEQ ID NO: 35 - RNA, 22 nt, synthetic construct
  source 1..22: mol_type = other RNA
cacagaacggttgtcggacttg

SEQ ID NO: 36 - RNA, 22 nt, synthetic construct
  source 1..22: mol_type = other RNA
aggtgaagtggtctggaaaggc

SEQ ID NO: 37 - RNA, 20 nt, synthetic construct
  source 1..20: mol_type = other RNA
gcaacagcatcttgcctgat

SEQ ID NO: 38 - RNA, 18 nt, synthetic construct
  source 1..18: mol_type = other RNA
gtgctactggtctcactt

SEQ ID NO: 39 - RNA, 20 nt, synthetic construct
  source 1..20: mol_type = other RNA
caaaacaccagtgtgaatta

SEQ ID NO: 40 - RNA, 20 nt, synthetic construct
  source 1..20: mol_type = other RNA
accatggtaatttcttgtga

SEQ ID NO: 41 - RNA, 20 nt, synthetic construct
  source 1..20: mol_type = other RNA
tgggtgtgaaccatgagaag

SEQ ID NO: 42 - RNA, 19 nt, synthetic construct
  source 1..19: mol_type = other RNA
gctaagcagttggtggtgc

SEQ ID NO: 43 - RNA, 22 nt, synthetic construct
  source 1..22: mol_type = other RNA
ggcaacagtagccacattggct

SEQ ID NO: 44 - RNA, 22 nt, synthetic construct
  source 1..22: mol_type = other RNA
gtctggatggtcatttcaccgc

SEQ ID NO: 45 - RNA, 22 nt, synthetic construct
  source 1..22: mol_type = other RNA
ggaccagaacacacgcttcctt

SEQ ID NO: 46 - RNA, 22 nt, synthetic construct
  source 1..22: mol_type = other RNA
ccgacattccatgttgaggctg

SEQ ID NO: 47 - RNA, 21 nt, synthetic construct
  source 1..21: mol_type = other RNA
cctctggtcttctggagtacc

SEQ ID NO: 48 - RNA, 20 nt, synthetic construct
  source 1..20: mol_type = other RNA
actccttctgtgactccagc

SEQ ID NO: 49 - RNA, 19 nt, synthetic construct
  source 1..19: mol_type = other RNA
atgagcacagaaagcatga

SEQ ID NO: 50 - RNA, 20 nt, synthetic construct
  source 1..20: mol_type = other RNA
agtagacagaagagcgtggt